(19) United States
(12) Patent Application Publication
(10) Pub. No.: US 2026/0124236 A1
(43) Pub. Date: May 7, 2026

(54) METHODS FOR TREATING EARLY ALZHEIMER’S DISEASE

(71) Applicant: CYCLO THERAPEUTICS, INC., Gainesville, FL (US)

(72) Inventors: Sharon H. Hrynkow, Annandale, VA (US); Jeffrey L. Tate, Newbury, FL (US); N. Scott Fine, Jupiter, FL (US)

(21) Appl. No.: 19/116,259

(22) PCT Filed: Sep. 26, 2023
(86) PCT No.: PCT/US2023/075118

§ 371 Date: Mar. 27, 2025

Related U.S. Application Data
(60) Provisional application No. 63/411,935, filed on Sep. 30, 2022.

(51) Int. Cl. A61K 31/724; A61K 31/13; A61K 31/27; A61K 31/427; A61K 31/433; A61K 31/445; A61K 31/549; A61K 31/55; A61K 39/395; A61K 45/06

(52) U.S. Cl. CPC A61K 31/724; A61K 31/13; A61K 31/27; A61K 31/427; A61K 31/433; A61K 31/445; A61K 31/549; A61K 31/55; A61K 39/3955; A61K 45/06

(57) ABSTRACT

Provided herein are methods for treating early Alzheimer's Disease using hydroxypropyl β-cyclodextrin compositions.

### CROSS-REFERENCE

This application claims priority to and the benefit of U.S. Provisional Patent Application No. 63/411,935, filed Sep. 30, 2022, the content of which is incorporated herein by reference in its entirety.

### BACKGROUND

Approximately 5.4 million Americans currently suffer from Alzheimer's disease, including early Alzheimer's disease, and this number is expected to rise to 13.8 million by 2050. In 2016, an estimated 700,000 Americans≥65 years old died with Alzheimer's disease. While United States (US) deaths from stroke, heart disease, and prostate cancer all decreased from 2000 to 2014, Alzheimer's disease-related deaths increased 89% over the same period. Total 2016 payments for health care, long-term care, and hospice services for people≥65 years old with dementia were estimated to be $236 billion. [Alzheimer's Association. 2016 Alzheimer's disease facts and figures. Alzheimers Dement. 2016; 12:459-509.]

More recently, with a better understanding of disease pathogenesis, drug development has shifted focus to limit, prevent, and mitigate amyloid-beta (Aβ) and tau accumulation. Numerous treatment strategies have been proposed for amyloid-based therapies that increase Aβ clearance or decrease Aβ aggregation, as well as targeted Aβ immunotherapy. To date, despite multiple attempts, these drugs have largely failed late-stage clinical trials, many of which enrolled patients in the later stages of dementia.

Doubts concerning the risk/benefit profile of anti-amyloid antibody therapies remain as they have been associated with potentially dangerous, albeit manageable, adverse reactions such as cerebral edema and microscopic hemorrhages. Like Aβ therapies, to date, tau-based therapies have yielded early hope but are currently in early phase studies and will not yield efficacy data for several years.

Cholesterol imbalance in Alzheimer's disease patients is well known, and significant research exists suggesting these imbalances are responsible for Aβ and tau accumulation. An increase in blood cholesterol is also associated with increased dementia risk, as plasma concentrations of cholesterol are significantly higher in Alzheimer's disease patients and patients with non-Alzheimer's-related dementia.

Both the generation and clearance of Aβ are regulated by cholesterol. Tau toxicity also depends upon cellular cholesterol levels. High cholesterol diets increase tau hyperphosphorylation. As the cholesterol concentration increases, so does the susceptibility of neurons to Aβ-dependent calpain activation. [Ferreira A, Bigio E H. Calpain-mediated tau cleavage: a mechanism leading to neurodegeneration shared by multiple tauopathies. Mol Med. 2011; 17(7-8):676-685.] Calpain activation cleaves tau and generates toxic fragments. These cleaved tau forms induce neuronal death, synapse loss, and/or behavioral deficits. Young neurons that have measurably less cholesterol than aged neurons, also contain less phosphorylated tau. However, when these same young neurons are loaded with cholesterol, both increases in phosphorylated tau as well as susceptibility to death are observed.

At present there are no approved therapies for treating early Alzheimer's disease through the modulation of cholesterol levels. As such, there is an unmet need to develop new therapies for the treatment of early Alzheimer's disease.

### SUMMARY

In one aspect, provided herein is a method of treating early Alzheimer's disease in a human patient in need thereof, the method generally comprising administering to the human patient an effective amount of a hydroxypropyl β-cyclodextrin composition.

In certain embodiments, the early Alzheimer's disease is Alzheimer's disease with mild cognitive impairment or mild Alzheimer's disease.

In certain embodiments, at the time of initiation of administration of the effective amount of the hydroxypropyl β-cyclodextrin composition the human patient exhibits progressive cognitive decline. In certain embodiments, at the time of initiation of administration of the effective amount of the hydroxypropyl β-cyclodextrin composition the human patient has exhibited progressive cognitive decline for at least about 1 year. In certain embodiments, at the time of initiation of administration of the effective amount of the hydroxypropyl β-cyclodextrin composition the human patient has a global Clinical Dementia Rating (CDR) scale score of about 0.5 and a CDR Memory Box score of about 0.5 or greater. In certain embodiments, at the time of initiation of administration of the effective amount of the hydroxypropyl β-cyclodextrin composition the human patient has a global Clinical Dementia Rating (CDR) scale score of between about 0.5 and about 1.0 and a CDR Memory Box score of about 0.5 or greater. In certain embodiments, at the time of initiation of administration of the effective amount of the hydroxypropyl β-cyclodextrin composition the human patient exhibits cerebral amyloid-beta (Aβ) pathology. In certain embodiments, at the time of initiation of administration of the effective amount of the hydroxypropyl β-cyclodextrin composition the human patient has a Mini-Mental State Examination-2: Standard Version (MMSE-2:SV) score of between about 20 and about 28.

In certain embodiments, the human patient has previously been administered a pro-cognitive drug and/or a symptomatic therapy for early Alzheimer's disease. In certain embodiments, the human patient has previously been administered an acetylcholinesterase inhibitor and/or memantine.

In certain embodiments, the human patient is at least 50 years old. In certain embodiments, the human patient is at least 60 years old. In certain embodiments, the human patient is at least 70 years old. In certain embodiments, the human patient is at least 80 years old.

In certain embodiments, administering an effective amount of the hydroxypropyl β-cyclodextrin composition comprises administering to the human patient about 500 mg/kg to about 2000 mg/kg of the hydroxypropyl β-cyclodextrin composition. In certain embodiments, administering an effective amount of the hydroxypropyl β-cyclodextrin composition comprises administering to the human patient by intravenous infusion about 500 mg/kg to about 2000 mg/kg of the hydroxypropyl β-cyclodextrin composition. In certain embodiments, administering an effective amount of the hydroxypropyl β-cyclodextrin composition comprises administering to the human patient by intravenous infusion about 500 mg/kg to about 2000 mg/kg of the hydroxypropyl β-cyclodextrin composition, every 28 days. In certain embodiments, administering an effective amount of the hydroxypropyl β-cyclodextrin composition comprises administering to the human patient by intravenous infusion about 500 mg/kg to about 2000 mg/kg of the hydroxypropyl β-cyclodextrin composition, every 28 days, for a period of at least about 24 weeks.

In certain embodiments, administering an effective amount of the hydroxypropyl β-cyclodextrin composition comprises administering to the human patient about 500 mg/kg to about 1000 mg/kg of the hydroxypropyl β-cyclodextrin composition. In certain embodiments, administering an effective amount of the hydroxypropyl β-cyclodextrin composition comprises administering to the human patient by intravenous infusion about 500 mg/kg to about 1000 mg/kg of the hydroxypropyl β-cyclodextrin composition. In certain embodiments, administering an effective amount of the hydroxypropyl β-cyclodextrin composition comprises administering to the human patient by intravenous infusion about 500 mg/kg to about 1000 mg/kg of the hydroxypropyl β-cyclodextrin composition, every 28 days. In certain embodiments, administering an effective amount of the hydroxypropyl β-cyclodextrin composition comprises administering to the human patient by intravenous infusion about 500 mg/kg to about 2000 mg/kg of the hydroxypropyl β-cyclodextrin composition, every 28 days, for a period of at least about 24 weeks.

In certain embodiments, the hydroxypropyl β-cyclodextrin composition is administered to the human patient by intravenous infusion over a period of at least about 4 hours. In certain embodiments, the hydroxypropyl β-cyclodextrin composition is administered to the human patient by intravenous infusion over a period of at least about 6.5 hours.

In certain embodiments, the hydroxypropyl β-cyclodextrin composition comprises about 25% (w/v) of a hydroxypropyl β-cyclodextrin.

In certain embodiments, the hydroxypropyl β-cyclodextrin composition comprises a mixture of two or more hydroxypropyl β-cyclodextrin species. In certain embodiments, each of the two or more hydroxypropyl β-cyclodextrin species has a different degree of hydroxypropylation of the β-cyclodextrin ring. In certain embodiments, the mixture of the two or more hydroxypropyl β-cyclodextrin species has a molar substitution value from about 0.59 to about 1.14. In certain embodiments, the mixture of the two or more hydroxypropyl β-cyclodextrin species has a molar substitution value from about 0.59 to about 0.8. In certain embodiments, the mixture of the two or more hydroxypropyl β-cyclodextrin species has a molar substitution value from about 0.8 to about 1.0. In certain embodiments, the hydroxypropyl β-cyclodextrin composition comprises about 0.2% w/w or less of unsubstituted β-cyclodextrin. In certain embodiments, the hydroxypropyl β-cyclodextrin composition comprises about 0.16% w/w or less of unsubstituted β-cyclodextrin. In certain embodiments, the hydroxypropyl β-cyclodextrin composition comprises about 2.5% (w/w) or less of propylene glycol.

In certain embodiments, the method further comprises administering a second therapeutic agent selected from the group consisting of donepezil, rivastigmine, galantamine, memantine, verubecestat, solanezumab, bapineuzumab, aducanumab, tideglusib, epothilone D, and ABBV-8E12.

In certain embodiments, the method further comprises administering a second therapeutic agent selected from the group consisting of a cholinesterase inhibitor, an NMDA receptor antagonist, a humanized antibody which targets tau protein, a humanized antibody which targets amyloid beta protein, and a BACE inhibitor.

In certain embodiments, the method further comprises administering a second therapeutic agent, wherein the second therapeutic agent is selected from any therapeutic agent set forth in Table 1.

### DETAILED DESCRIPTION

The present disclosure provides, in part, a method for treating early Alzheimer's disease in a human patient in need thereof. The method generally comprises administering to the human patient an effective amount of a hydroxypropyl β-cyclodextrin composition. The hydroxypropyl β-cyclodextrin compositions described herein may be administered to the human patient intravenously. The methods described herein may stabilize early Alzheimer's disease progression and/or reverse key features of the disease (e.g., impaired cognitive function) in the human patient.

### Definitions

To facilitate an understanding of the present invention, a number of terms and phrases are defined below.

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

Throughout the description, where compositions and kits are described as having, including, or comprising specific components, it is contemplated that, additionally, there are compositions and kits of the present invention that consist essentially of, or consist of, the recited components.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the order of steps or order for performing certain actions is immaterial so long as the present invention remain operable. Moreover, two or more steps or actions may be conducted simultaneously.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

The articles “a” and “an” are used in this disclosure to refer to one or more than one (i.e., to at least one) of the grammatical object of the article, unless the context is inappropriate. By way of example, “an element” means one element or more than one element.

The term “and/or” is used in this disclosure to mean either “and” or “or” unless indicated otherwise.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

As used herein, the term “about” means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%, 5%, 3%, 2%, or 1%.

“Individual,” “patient,” and “subject” are used interchangeably and include any animal, including mammals, e.g., mice, rats, other rodents, rabbits, dogs, cats, swine, cattle, sheep, horses, or primates, including humans.

The terms “treat”, “treating” or “treatment” includes any effect, for example, lessening, reducing, modulating, ameliorating or eliminating, that results in the improvement of the condition, disease, disorder, and the like, or ameliorating a symptom thereof. Treating can be curing, improving, or at least partially ameliorating the disorder. In certain embodiments, treating is curing the disease.

“Pharmaceutically acceptable” includes molecular entities and formulations that do not produce an adverse, allergic or other untoward reaction when administered to an animal, or a human, as appropriate. For human administration, preparations should meet sterility, pyrogenicity, and general safety and purity standards as required by FDA Office of Biologics standards.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water (e.g., water for injection (WFI)), NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethylcellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The pharmaceutical formulations of the disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods of the disclosure is desirably a mammal in which treatment of early Alzheimer's disease is desired.

As used herein, “pharmaceutical composition” or “pharmaceutical formulation” may refer to the combination of an active agent with a carrier, inert or active, making the composition especially suitable for diagnostic or therapeutic use in vivo or ex vivo.

As used herein, “effective amount” or “therapeutically-effective amount” may refer to the amount of a compound or composition (e.g., a compound or composition of the present invention) sufficient to effect beneficial or desired results. An effective amount can be administered in one or more administrations, applications or dosages and is not intended to be limited to a particular formulation or administration route.

As used herein, “administer” or “administering” may refer to oral administration, administration as a suppository, topical contact, intravenous, parenteral, intraperitoneal, intramuscular, intralesional, intrathecal, intracranial, intracerebroventricular, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., anti-cancer agent, chemotherapeutic, or treatment for a neurodegenerative disease). The compound of the invention can be administered alone or can be co-administered to the patient. Coadministration is meant to include simultaneous or sequential administration of the compound individually or in combination (more than one compound or agent). Thus, the preparations can also be combined, when desired, with other active substances (e.g., to reduce metabolic degradation).

As used herein, “hydroxypropyl β-cyclodextrin species”, “β-cyclodextrin species”, or “β-cyclodextrins” may refer to a β-cyclodextrin molecule with a unique chemical composition and/or chemical structure. For example, a hydroxypropyl β-cyclodextrin species of the present invention may have unique properties including, but not limited to, average number of hydroxypropyl groups per β-cyclodextrin molecule, molar substitution value, distribution of hydroxypropyl groups, degree of distribution of hydroxypropyl groups, or any combination thereof. In certain embodiments, the hydroxypropyl β-cyclodextrin may be referred to by the trademark name Trappsol® Cyclo™. The terms “hydroxypropyl β-cyclodextrin”, “hydroxypropyl beta-cyclodextrin”, “hydroxypropyl-beta-cyclodextrin”, “HPβCD”, “2-hydroxypropyl-beta-cyclodextrin”, “2-hydroxypropyl beta-cyclodextrin” and “2-hydroxypropyl β-cyclodextrin” may be used interchangeably herein. The terms “β-cyclodextrin” and “beta-cyclodextrin” may be used interchangeably herein.

As used herein, “substituted at one or more hydroxyl positions by hydroxypropyl groups” may refer to replacement of the hydrogen of one or more hydroxyl groups of a beta-cyclodextrin molecule with a hydroxypropyl group or a hydroxypropyl oligomer. For instance, “substituted at one or more hydroxyl positions by hydroxypropyl groups” can refer to an insertion of one or more CH₂CH(CH₃)O-substituents within one or more O—H bonds on a beta-cyclodextrin molecule resulting in one or more ether linkages.

As used herein, the “cognitive function” or “cognitive functioning” of a subject may be defined as an intellectual activity or process. Examples of intellectual activities or processes include, but are not limited to, attention, processing speed, learning and memory, executive function, verbal fluency and working memory. For example, a hydroxypropyl-beta-cyclodextrin composition of the present invention may improve cognitive function if it improves one or more intellectual activities or processes in a subject with early Alzheimer's disease.

For patients with early Alzheimer's disease, the Food and Drug Administration (FDA) considers the Clinical Dementia Rating scale Sum of Boxes (CDR-SB) as an example of a tool to assess disease progression. The Clinical Dementia Rating (CDR) is obtained through semi-structured interviews of patients and informants, and cognitive functioning is rated in 6 domains of functioning: memory, orientation, judgment and problem solving, community affairs, home and hobbies, and personal care. Each domain is rated on a 5-point scale of functioning as follows: 0 (no impairment), 0.5 (questionable impairment), 1 (mild impairment), 2 (moderate impairment), and 3 (severe impairment). Personal care is scored on a 4-point scale without a 0.5 rating available. The global CDR score is computed via an algorithm. The CDR-SB score is obtained by summing each of the domain box scores, with scores ranging from 0 to 18. The CDR demonstrates good reliability and has been validated against neuropathologic finding.

As used herein, “amyloid-beta pathology” or “Aβ pathology” may refer to patients who have evidence of amyloid-beta pathology assessed by one or more methodologies or tests validated to predict the likelihood of amyloid plaque burden, or quantified by PET imaging, cerebrospinal fluid analysis and/or MRI. The Precivity AD blood test (C₂N Diagnostics) is a test for the detection of AD pathology and uses mass spectrometry to measure proteins in the blood (derived from a single blood sample) that indicate the probability of amyloid deposits in the brain, as measured by amyloid PET scans. The Precivity AD test quantifies plasma concentrations of amyloid-beta 42 and 40 (Aβ42 and Aβ40) and determines the presence of Apolipoprotein E proteotype (equivalent to ApoE genotype) specific peptides. These parameters are used to calculate a patient's Aβ42/40 ratio and to determine his/her ApoE genotype. The Aβ42/40 ratio and ApoE genotype, as well as the patient's age, are incorporated into the test's statistical algorithm to estimate an APS (e.g., the test result). The APS represents the estimated probability from 0 (low probability) to 100 (high probability) that the patient is currently amyloid positive on amyloid PET imaging based on his or her Aβ42/40 ratio, age, and ApoE genotype. A positive amyloid PET scan is consistent with the presence of amyloid plaques and an AD diagnosis. In addition to the APS, the test report will also include the patient's Aβ42/40 ratio and ApoE genotype profile. While each of these parameters can individually predict amyloid PET scan status, their use in combination with the patient's age were included in modeling to derive the APS, which is associated with superior risk prediction. Therefore, APS is the most important result. The APS score will generally classify the patient in 1 of 3 categories: low, intermediate, or high, which should be interpreted as follows:
Low APS (0-36): a low score is consistent with a negative amyloid PET scan result and, thus, a low likelihood of amyloid plaques. Absence of amyloid plaques is inconsistent with an using one of the approved neuroimaging agents as the tracer AD diagnosis and indicates other causes of cognitive symptoms should be investigated.
Intermediate APS (37-57): an intermediate score does not distinguish between the presence or absence of amyloid plaques and indicates further diagnostic evaluation may be needed to assess the underlying cause(s) for the patient's cognitive symptoms.
High APS (58-100): a high score is consistent with a positive amyloid PET scan result and, thus, a high likelihood of amyloid plaques. Presence of amyloid plaques is consistent with an AD diagnosis in someone who has cognitive decline, but alone is insufficient for a final diagnosis; clinical presentation and other factors should be considered along with APS. Combination of a positive PrecivityAD blood test, other eligibility criteria including, cognitive test results and quantification of amyloid burden evidenced by either Positron Emission Tomography Amyloid Imaging, Cerebrospinal Fluid analysis form the basis for evidence of amyloid burden. The Aβ-PET scan (using one of the approved neuroimaging agents as the tracer) will result images that will be designated as positive or negative based upon a comparison of radioactivity in cortical gray matter with activity in adjacent white matter in regions of interest The amyloid The CSF analysis will evaluate the ratio of Aβ42, Aβ40 and Aβ42/Aβ40 ratio. Other biomarkers such as, but limited to, phosphorylated tau, total tau, and NfL.

As used herein, the terms “Mini-Mental State Examination-2: Standard Version” or “MMSE-2:SV” may refer to a widely used test of cognitive function among the elderly. It includes tests of orientation, attention, memory, language, and visual-spatial skills, and the results are assigned a number score. [Folstein M F, Folstein S E, White T, Messer M A. MMSE-2® Mini-Mental State Examination, 2nd Edition™ User's Manual by Marshal F. Available at https://www.parinc.com/Products/Pkey/238. Accessed 7 Oct. 2021.] This test may be used to screen for cognitive impairment, to estimate the severity of cognitive impairment at a given point in time, to track progression of cognitive impairment associated with dementia, and to document an individual's response to treatment. [Folstein M F, Folstein S E, McHugh P R. Mini-Mental State Examination. PAR. Available at https://www.parinc.com/Products/Pkey/237. Accessed 30 Apr. 2020.]

As used herein, the term “molar substitution”, or “MS”, refers to the number of hydroxypropyl groups per anhydroglucose unit in the mixture of β-cyclodextrins, and may be determined according to the procedures set forth in the USP monograph on Hydroxypropyl Betadex (USP NF 2015) (“USP Hydroxypropyl Betadex monograph”), incorporated herein by reference in its entirety. In this disclosure, the term “average molar substitution”, or “MSₐ”, is used synonymously with “MS” as that term is used in the USP Hydroxypropyl Betadex monograph, and the term “glucose unit” is used as a synonym for “anhydroglucose unit”, as that term is used in the USP Hydroxypropyl Betadex monograph.

### Hydroxypropyl β-Cyclodextrins

Cyclodextrins are naturally occurring cyclic oligosaccharides derived from the enzymatic conversion of starch and can also be synthetically manufactured. Cyclodextrins are composed of a variable number of glucopyranose units that may form a hollow cone-like toroid structure consisting of a hydrophobic cavity and hydrophilic exterior. The hollow cone-like toroid structure may also be referred to as “beta-cyclodextrin ring”. The number of glucopyranose determines the cavity size and nomenclature of cyclodextrins, with the most common consisting of six, seven, or eight glucopyranose units and named α-, β-, and γ-cyclodextrin, respectively. The unique structure of cyclodextrins allows them to form water-soluble complexes with otherwise insoluble hydrophobic compounds. This property of cyclodextrins has led to their application as drug delivery vehicles to improve solubility, stability, and bioavailability of many pharmacologically active agents. Hydroxypropyl-beta-cyclodextrin (HPβCD), which, as stated above, is also known as and may be referred to herein as 2-hydroxypropyl-beta-cyclodextrin, is a highly soluble, chemically modified synthetic derivative of beta-cyclodextrin. HPβCD is one of the most commonly used and least toxic derivatives of a naturally occurring cyclodextrin for drug delivery.

In one aspect, provided herein are hydroxypropyl β-cyclodextrin compositions for the treatment of early Alzheimer's disease in a subject in need thereof. In certain embodiments, the subject is a human patient.

In certain embodiments, a hydroxypropyl β-cyclodextrin composition described herein can be a mixture of two or more hydroxypropyl-beta-cyclodextrin species. In some embodiments, the mixture of hydroxypropyl β-cyclodextrin species comprises a mixture of β-cyclodextrin molecules substituted at one or more hydroxyl positions by hydroxypropyl groups.

In certain embodiments, a hydroxypropyl β-cyclodextrin composition described herein comprise a mixture of two or more hydroxypropyl β-cyclodextrin species. In some embodiments, the hydroxypropyl β-cyclodextrin composition comprises a mixture of 2, 3, 4, 5, 6, 7, 8, 9, 10, 11, 12, 13, 14, 15, 16, 17, 18, 19, or 20 hydroxypropyl β-cyclodextrin species. In some embodiments, each of the two or more hydroxypropyl β-cyclodextrin species in the mixture has a different degree of hydroxypropylation of the β-cyclodextrin ring.

In certain embodiments, a hydroxypropyl β-cyclodextrin species in the mixture comprises glucose units of the structure:

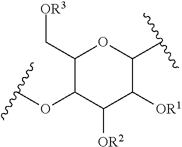

wherein R¹, R², and R³, independently for each occurrence, are H or HP, wherein HP comprises one or more hydroxypropyl groups.

In certain embodiments, HP comprises 1, 2, 3, 4, 5, 6, 7, 8, 9, or 10 hydroxypropyl groups. In some embodiments, HP comprises one hydroxypropyl group. In certain embodiments, HP consists of 1, 2, 3, 4, 5, 6, 7, 8, 9, or 10 hydroxypropyl groups. In some embodiments, HP consists of one hydroxypropyl group.

In certain embodiments, the average number of occurrences of HP per β-cyclodextrin ring is about 3 to about 7, about 3 to about 6, about 3 to about 5, about 3 to about 4, about 4 to about 7, about 4 to about 6, about 4 to about 5, about 5 to about 7, about 5 to about 6, or about 6 to about 7. In certain embodiments, the average number of occurrences of HP per beta-cyclodextrin ring is about 3, about 4, about 5, about 6, or about 7.

In certain embodiments, the total occurrences of R³=HP are greater than the total occurrences of either R¹=HP or R²=HP. In certain embodiments, the total occurrences of R³=HP are greater than the total combined occurrences of R¹=HP and R²=HP.

In certain embodiments, at least about 5%, at least about 10%, at least about 15%, at least about 20%, at least about 25%, at least about 30%, at least about 35%, at least about 40%, or at least about 45% of the total combined occurrences of R¹ and R² are HP.

In some embodiments, not more than about 50%, not more than about 55%, not more than about 60%, not more than about 65%, not more than about 70%, not more than about 75%, not more than about 80%, not more than about 85%, not more than about 90%, or not more than about 95% of the total combined occurrences of R¹ and R² are HP.

In certain embodiments, the percentage of R¹ and R² combined that are HP ranges from about 5% to about 95%, about 10% to about 95%, about 15% to about 95%, about 20% to about 95%, about 25% to about 95%, about 30% to about 95%, about 35% to about 95%, about 40% to about 95%, about 45% to about 95%, about 50% to about 95%, about 55% to about 95%, about 60% to about 95%, about 65% to about 95%, about 70% to about 95%, about 75% to about 95%, about 80% to about 95%, about 85% to about 95%, about 90% to about 95%; about 5% to about 90%, about 10% to about 90%, about 15% to about 90%, about 20% to about 90%, about 25% to about 90%, about 30% to about 90%, about 35% to about 90%, about 40% to about 90%, about 45% to about 90%, about 50% to about 90%, about 55% to about 90%, about 60% to about 90%, about 65% to about 90%, about 70% to about 90%, about 75% to about 90%, about 80% to about 90%, about 85% to about 90%; about 5% to about 85%, about 10% to about 85%, about 15% to about 85%, about 20% to about 85%, about 25% to about 85%, about 30% to about 85%, about 35% to about 85%, about 40% to about 85%, about 45% to about 85%, about 50% to about 85%, about 55% to about 85%, about 60% to about 85%, about 65% to about 85%, about 70% to about 85%, about 75% to about 85%, about 80% to about 85%; about 5% to about 80%, about 10% to about 80%, about 15% to about 80%, about 20% to about 80%, about 25% to about 80%, about 30% to about 80%, about 35% to about 80%, about 40% to about 80%, about 45% to about 80%, about 50% to about 80%, about 55% to about 80%, about 60% to about 80%, about 65% to about 80%, about 70% to about 80%, about 75% to about 80%; about 5% to about 75%, about 10% to about 75%, about 15% to about 75%, about 20% to about 75%, about 25% to about 75%, about 30% to about 75%, about 35% to about 75%, about 40% to about 75%, about 45% to about 75%, about 50% to about 75%, about 55% to about 75%, about 60% to about 75%, about 65% to about 75%, about 70% to about 75%; about 5% to about 70%, about 10% to about 70%, about 15% to about 70%, about 20% to about 70%, about 25% to about 70%, about 30% to about 70%, about 35% to about 70%, about 40% to about 70%, about 45% to about 70%, about 50% to about 70%, about 55% to about 70%, about 60% to about 70%, about 65% to about 70%; about 5% to about 65%, about 10% to about 65%, about 15% to about 65%, about 20% to about 65%, about 25% to about 65%, about 30% to about 65%, about 35% to about 65%, about 40% to about 65%, about 45% to about 65%, about 50% to about 65%, about 55% to about 65%, about 60% to about 65%; about 5% to about 60%, about 10% to about 60%, about 15% to about 60%, about 20% to about 60%, about 25% to about 60%, about 30% to about 60%, about 35% to about 60%, about 40% to about 60%, about 45% to about 60%, about 50% to about 60%, about 55% to about 60%; about 5% to about 55%, about 10% to about 55%, about 15% to about 55%, about 20% to about 55%, about 25% to about 55%, about 30% to about 55%, about 35% to about 55%, about 40% to about 55%, about 45% to about 55%, about 50% to about 55%; about 5% to about 50%, about 10% to about 50%, about 15% to about 50%, about 20% to about 50%, about 25% to about 50%, about 30% to about 50%, about 35% to about 50%, about 40% to about 50%, about 45% to about 50%; about 5% to about 45%, about 10% to about 45%, about 15% to about 45%, about 20% to about 45%, about 25% to about 45%, about 30% to about 45%, about 35% to about 45%, about 40% to about 45%; about 5% to about 40%, about 10% to about 40%, about 15% to about 40%, about 20% to about 40%, about 25% to about 40%, about 30% to about 40%, about 35% to about 40%; about 5% to about 35%, about 10% to about 35%, about 15% to about 35%, about 20% to about 35%, about 25% to about 35%, about 30% to about 35%; about 5% to about 30%, about 10% to about 30%, about 15% to about 30%, about 20% to about 30%, about 25% to about 30%; about 5% to about 25%, about 10% to about 25%, about 15% to about 25%, about 20% to about 25%; about 5% to about 20%, about 10% to about 20%, about 15% to about 20%; about 5% to about 15%, about 10% to about 15%; or about 5% to about 10%.

In certain embodiments, at least about 5%, at least about 10%, at least about 15%, at least about 20%, at least about 25%, at least about 30%, at least about 35%, at least about 40%, at least about 45%, or at least about 50% of occurrences of R³ are HP.

In certain embodiments, not more than about 55%, not more than about 60%, not more than about 65%, not more than about 70%, not more than about 75%, not more than about 80%, not more than about 85%, not more than about 90%, or not more than about 95% of occurrences of R³ are HP.

In certain embodiments, the percentage of occurrence of R³ that are HP ranges from about 20% to about 90%, about 25% to about 90%, about 30% to about 90%, about 35% to about 90%, about 40% to about 90%, about 45% to about 90%, about 50% to about 90%, about 55% to about 90%, about 60% to about 90%, about 65% to about 90%, about 70% to about 90%, about 75% to about 90%, about 80% to about 90%, about 85% to about 90%, about 20% to about 85%, about 25% to about 85%, about 30% to about 85%, about 35% to about 85%, about 40% to about 85%, about 45% to about 85%, about 50% to about 85%, about 55% to about 85%, about 60% to about 85%, about 65% to about 85%, about 70% to about 85%, about 75% to about 85%, about 80% to about 85%, about 20% to about 80%, about 25% to about 80%, about 30% to about 80%, about 35% to about 80%, about 40% to about 80%, about 45% to about 80%, about 50% to about 80%, about 55% to about 80%, about 60% to about 80%, about 65% to about 80%, about 70% to about 80%, about 75% to about 80%, about 20% to about 75%, about 25% to about 75%, about 30% to about 75%, about 35% to about 75%, about 40% to about 75%, about 45% to about 75%, about 50% to about 75%, about 55% to about 75%, about 60% to about 75%, about 65% to about 75%, about 70% to about 75%, about 20% to about 70%, about 25% to about 70%, about 30% to about 70%, about 35% to about 70%, about 40% to about 70%, about 45% to about 70%, about 50% to about 70%, about 55% to about 70%, about 60% to about 70%, about 65% to about 70%, about 20% to about 65%, about 25% to about 65%, about 30% to about 65%, about 35% to about 65%, about 40% to about 65%, about 45% to about 65%, about 50% to about 65%, about 55% to about 65%, about 60% to about 65%, about 20% to about 60%, about 25% to about 60%, about 30% to about 60%, about 35% to about 60%, about 40% to about 60%, about 45% to about 60%, about 50% to about 60%, about 55% to about 60%, about 20% to about 55%, about 25% to about 55%, about 30% to about 55%, about 35% to about 55%, about 40% to about 55%, about 45% to about 55%, about 50% to about 55%, about 20% to about 50%, about 25% to about 50%, about 30% to about 50%, about 35% to about 50%, about 40% to about 50%, about 45% to about 50%, about 20% to about 45%, about 25% to about 45%, about 30% to about 45%, about 35% to about 45%, about 40% to about 45%, about 5% to about 40%, about 10% to about 40%, about 15% to about 40%, about 20% to about 40%, about 25% to about 40%, about 30% to about 40%, about 35% to about 40%, about 20% to about 35%, about 25% to about 35%, about 30% to about 35%, about 20% to about 30%, about 25% to about 30%, or about 20% to about 25%.

In certain embodiments, at least about 70%, at least about 75%, at least about 80%, at least about 85%, at least about 90%, or at least about 95%, of the β-cyclodextrins collectively have an average number of occurrences of HP per β-cyclodextrin of about 4 to about 7, about 4 to about 6, about 4 to about 5, about 5 to about 7, about 5 to about 6, or about 6 to about 7.

In some embodiments, the percentage of β-cyclodextrins that collectively have an average number of occurrences of HP per-cyclodextrin of about 4 to about 7, about 4 to about 6, about 4 to about 5, about 5 to about 7, about 5 to about 6, or about 6 to about 7, ranges from about 50% to about 99%, about 55% to about 99%, about 60% to about 99%, about 65% to about 99%, about 70% to about 99%, about 75% to about 99%, about 80% to about 99%, about 85% to about 99%, about 90% to about 99%, about 95% to about 99%, about 50% to about 97%, about 55% to about 97%, about 60% to about 97%, about 65% to about 97%, about 70% to about 97%, about 75% to about 97%, about 80% to about 97%, about 85% to about 97%, about 90% to about 97%, about 95% to about 97%, about 50% to about 95%, about 55% to about 95%, about 60% to about 95%, about 65% to about 95%, about 70% to about 95%, about 75% to about 95%, about 80% to about 95%, about 85% to about 95%, about 90% to about 95%, about 50% to about 90%, about 55% to about 90%, about 60% to about 90%, about 65% to about 90%, about 70% to about 90%, about 75% to about 90%, about 80% to about 90%, about 85% to about 90%, about 50% to about 85%, about 55% to about 85%, about 60% to about 85%, about 65% to about 85%, about 70% to about 85%, about 75% to about 85%, about 80% to about 85%, about 50% to about 80%, about 55% to about 80%, about 60% to about 80%, about 65% to about 80%, about 70% to about 80%, about 75% to about 80%, about 50% to about 75%, about 55% to about 75%, about 60% to about 75%, about 65% to about 75%, about 70% to about 75%, about 50% to about 70%, about 55% to about 70%, about 60% to about 70%, about 65% to about 70%, about 50% to about 65%, about 55% to about 65%, about 60% to about 65%, about 50% to about 60%, about 55% to about 60%, or about 50% to about 55%.

As used herein, the “degree of substitution” or “DS” may refer to the total number of hydroxypropyl groups substituted directly or indirectly on a β-cyclodextrin molecule. For example, a β-cyclodextrin molecule containing glucose units, each of which is substituted with one hydroxypropyl group, has a DS=7. In another example, a β-cyclodextrin molecule in which only one of the seven glucose units is substituted with a hydroxypropyl group, and that hydroxypropyl group is itself substituted with another hydroxypropyl group (e.g., a beta-cyclodextrin with a single occurrence of HP that comprises two hydroxypropyl groups), has a DS=2.

As used herein, the “average number of hydroxypropyl groups per beta-cyclodextrin,” also known as an “average degree of substitution,” “average DS,” or “DSₐ,” may refer to the total number of hydroxypropyl groups in a population of β-cyclodextrins divided by the number of β-cyclodextrin molecules. In an illustrative example, an equal parts mixture of β-cyclodextrins containing glucose units that are each substituted with one hydroxypropyl group and β-cyclodextrins containing glucose units that are each substituted with two hydroxypropyl groups has a DSₐ=10.5 (average of equal parts β-cyclodextrins with DS=7 and DS=14). In another illustrative example, a mixture of 33.3% β-cyclodextrins in which only one of the seven glucose units is substituted with a hydroxypropyl group (DS=1) and 66.7% β-cyclodextrins containing glucose units that are each substituted with one hydroxypropyl group (DS=7) has a DSₐ=5.0. The DSₐ may be determined by multiplying the molar substitution by 7. As used herein, DSₐ is used synonymously with “degree of substitution” as that term is defined in the USP Hydroxypropyl Betadex monograph.

In certain embodiments, the hydroxypropyl β-cyclodextrin compositions of the present invention comprise a mixture of unsubstituted β-cyclodextrin molecules and β-cyclodextrin species substituted at one or more hydroxyl positions by hydroxypropyl groups, wherein the mixture has an average number of hydroxypropyl groups per β-cyclodextrin molecule (DSₐ) of about 3 to about 7, about 3 to about 6, about 3 to about 5, about 3 to about 4, about 4 to about 7, about 4 to about 6, about 4 to about 5, about 5 to about 7, about 5 to about 6, or about 6 to about 7.

The distribution of the degree of substitution within the hydroxypropyl β-cyclodextrin compositions of the present invention comprising a mixture of unsubstituted β-cyclodextrin molecules and β-cyclodextrin species substituted at one or more hydroxyl positions by hydroxypropyl groups can vary. For example, an equal parts mixture of β-cyclodextrins containing glucose units each of which is substituted with one hydroxypropyl group and β-cyclodextrins containing glucose units each of which is substituted with two hydroxypropyl groups has a DSₐ=10.5 (average of equal parts β-cyclodextrins with DS=7 and DS=14). Although DSₐ=10.5, in this example there are no β-cyclodextrins having DS=10 or DS=11 within the mixture. In other cases, the majority of β-cyclodextrin species within the mixture of β-cyclodextrins have DS that are close to the DSₐ.

In certain embodiments, at least about 50%, about 55%, about 60%, about 65%, about 70%, about 75%, about 80%, about 85%, about 90%, about 95%, or about 97%, of the β-cyclodextrins within the mixture have a DS within DSₐ±Xσ, wherein σ is the standard deviation, and X is 1, 2, or 3.

In certain embodiments, at least about 50% of the β-cyclodextrins within the mixture have a DS within DSₐ±10. In some embodiments, about 55%, about 60%, about 65%, about 70%, about 75%, about 80%, about 85%, about 90%, about 95%, or about 97%, of the β-cyclodextrins within the mixture have a DS within DSₐ±1σ.

In certain embodiments, at least about 50% of the β-cyclodextrins within the mixture have a DS within DSₐ±20. In some embodiments, about 55%, about 60%, about 65%, about 70%, about 75%, about 80%, about 85%, about 90%, about 95%, or about 97%, of the β-cyclodextrins within the mixture have a DS within DSₐ±2σ.

In certain embodiments, at least about 50% of the β-cyclodextrins within the mixture have a DS within DSₐ±30. In some embodiments, about 55%, about 60%, about 65%, about 70%, about 75%, about 80%, about 85%, about 90%, about 95%, or about 97%, of the β-cyclodextrins within the mixture have a DS within DSₐ±30.

In certain embodiments, at least about 50% of the β-cyclodextrins have a DS within DSₐ±1. In some embodiments, about 55%, about 60%, about 65%, about 70%, about 75%, about 80%, about 85%, about 90%, about 95%, or about 97%, of the β-cyclodextrins have a DS within DSₐ±1.

In certain embodiments, at least about 50% of the β-cyclodextrins have a DS within DSₐ±0.8. In some embodiments, about 55%, about 60%, about 65%, about 70%, about 75%, about 80%, about 85%, about 90%, about 95%, or about 97%, of the β-cyclodextrins have a DS within DSₐ±0.8.

In certain embodiments, at least about 50% of the β-cyclodextrins have a DS within DSₐ±0.6. In some embodiments, about 55%, about 60%, about 65%, about 70%, about 75%, about 80%, about 85%, about 90%, about 95%, or about 97%, of the β-cyclodextrins have a DS within DSₐ=0.6.

In certain embodiments, at least about 50% of the β-cyclodextrins have a DS within DSₐ±0.5. In some embodiments, about 55%, about 60%, about 65%, about 70%, about 75%, about 80%, about 85%, about 90%, about 95%, or about 97%, of the β-cyclodextrins have a DS within DSₐ±0.5.

In certain embodiments, at least about 50% of the β-cyclodextrins have a DS within DSₐ=0.4. In some embodiments, about 55%, about 60%, about 65%, about 70%, about 75%, about 80%, about 85%, about 90%, about 95%, or about 97%, of the β-cyclodextrins have a DS within DSₐ±0.4.

In certain embodiments, at least about 50% of the β-cyclodextrins have a DS within DSₐ±0.3. In some embodiments, about 55%, about 60%, about 65%, about 70%, about 75%, about 80%, about 85%, about 90%, about 95%, or about 97%, of the β-cyclodextrins have a DS within DSₐ±0.3.

In certain embodiments, at least about 50% of the β-cyclodextrins have a DS within DSₐ±0.2. In some embodiments, about 55%, about 60%, about 65%, about 70%, about 75%, about 80%, about 85%, about 90%, about 95%, or about 97%, of the β-cyclodextrins have a DS within DSₐ±0.2.

In certain embodiments, at least about 50% of the β-cyclodextrins have a DS within DSₐ=0.1. In some embodiments, about 55%, about 60%, about 65%, about 70%, about 75%, about 80%, about 85%, about 90%, about 95%, or about 97%, of the β-cyclodextrins have a DS within DSₐ±0.1.

The number of hydroxypropyl groups per anhydroglucose unit in the mixture of β-cyclodextrins is known as the “molar substitution”, or “MS”, and may be determined according to the procedures set forth in the USP monograph on Hydroxypropyl Betadex (USP NF 2015) (“USP Hydroxypropyl Betadex monograph”), incorporated herein by reference in its entirety. In this disclosure, the term “average molar substitution”, or “MSₐ”, is used synonymously with “MS” as that term is used in the USP Hydroxypropyl Betadex monograph, and the term “glucose unit” is used as a synonym for “anhydroglucose unit”, as that term is used in the USP Hydroxypropyl Betadex monograph.

In some embodiments, the MS of the mixture of hydroxypropyl β-cyclodextrin species is from about 0.51 to about 0.8, about 0.51 to about 0.77, about 0.51 to about 0.75, about 0.51 to about 0.73, about 0.51 to about 0.71, about 0.51 to about 0.69, about 0.51 to about 0.67, about 0.51 to about 0.65, about 0.51 to about 0.63, about 0.51 to about 0.61, about 0.51 to about 0.59, about 0.51 to about 0.57, about 0.51 to about 0.55, about 0.51 to about 0.53, 0.53 to about 0.8, 0.53 to about 0.77, 0.53 to about 0.75, about 0.53 to about 0.73, about 0.53 to about 0.71, about 0.53 to about 0.69, about 0.53 to about 0.67, about 0.53 to about 0.65, about 0.53 to about 0.63, about 0.53 to about 0.61, about 0.53 to about 0.59, about 0.53 to about 0.57, about 0.53 to about 0.55, 0.55 to about 0.8, 0.55 to about 0.77, 0.55 to about 0.75, about 0.55 to about 0.73, about 0.55 to about 0.71, about 0.55 to about 0.69, about 0.55 to about 0.67, about 0.55 to about 0.65, about 0.55 to about 0.63, about 0.55 to about 0.61, about 0.55 to about 0.59, about 0.55 to about 0.57, 0.57 to about 0.8, 0.57 to about 0.77, 0.57 to about 0.75, about 0.57 to about 0.73, about 0.57 to about 0.71, about 0.57 to about 0.69, about 0.57 to about 0.67, about 0.57 to about 0.65, about 0.57 to about 0.63, about 0.57 to about 0.61, about 0.57 to about 0.59, 0.59 to about 0.8, 0.59 to about 0.77, 0.59 to about 0.75, about 0.59 to about 0.73, about 0.59 to about 0.71, about 0.59 to about 0.69, about 0.59 to about 0.67, about 0.59 to about 0.65, about 0.59 to about 0.63, about 0.59 to about 0.61, 0.61 to about 0.8, 0.61 to about 0.77, 0.61 to about 0.75, about 0.61 to about 0.73, about 0.61 to about 0.71, about 0.61 to about 0.69, about 0.61 to about 0.67, about 0.61 to about 0.65, about 0.61 to about 0.63, 0.63 to about 0.8, 0.63 to about 0.77, 0.63 to about 0.75, about 0.63 to about 0.73, about 0.63 to about 0.71, about 0.63 to about 0.69, about 0.63 to about 0.67, about 0.63 to about 0.65, 0.65 to about 0.8, 0.65 to about 0.77, 0.65 to about 0.75, about 0.65 to about 0.73, about 0.65 to about 0.71, about 0.65 to about 0.69, about 0.65 to about 0.67, 0.67 to about 0.8, 0.67 to about 0.77, 0.67 to about 0.75, about 0.67 to about 0.73, about 0.67 to about 0.71, about 0.67 to about 0.69, 0.69 to about 0.8, 0.69 to about 0.77, 0.69 to about 0.75, about 0.69 to about 0.73, about 0.69 to about 0.71, 0.71 to about 0.8, 0.71 to about 0.77, 0.71 to about 0.75, about 0.71 to about 0.73, about 0.73 to about 0.8, about 0.73 to about 0.77, about 0.73 to about 0.75, about 0.75 to about 0.8, about 0.73 to about 0.77, or about 0.77 to about 0.8. In some embodiments, the MS of the mixture of hydroxypropyl β-cyclodextrin species is from about 0.59 to about 0.73. In some embodiments, the MS of the mixture of hydroxypropyl β-cyclodextrin species is from about 0.59 to about 0.8.

In certain embodiments, the mixture of the two or more hydroxypropyl β-cyclodextrin species has a molar substitution (MS) value from about 0.5 to about 1.2, about 0.6 to about 1.2, about 0.7 to about 1.2, about 0.8 to about 1.2, about 0.9 to about 1.2, about 1.0 to about 1.2, about 1.1 to about 1.2, about 0.5 to about 1.1, about 0.5 to about 1.0, about 0.5 to about 0.9, about 0.5 to about 0.8, about 0.5 to about 0.7, about 0.5 to about 0.6, about 0.6 to about 1.1, about 0.6 to about 1.0, about 0.6 to about 0.9, about 0.6 to about 0.8, about 0.6 to about 0.7, about 0.7 to about 1.1, about 0.7 to about 1.0, about 0.7 to about 0.9, about 0.7 to about 0.8, about 0.8 to about 1.1, about 0.8 to about 1.0, about 0.8 to about 0.9, about 0.9 to about 1.1, about 0.9 to about 1.0, or about 1.0 to about 1.1. In certain embodiments, the mixture of the two or more hydroxypropyl β-cyclodextrin species has a molar substitution value from about 0.59 to about 1.14. In certain embodiments, the mixture of the two or more hydroxypropyl β-cyclodextrin species has a molar substitution value from about 0.59 to 0.73. In certain embodiments, the mixture of the two or more hydroxypropyl β-cyclodextrin species has a molar substitution value from about 0.59 to 0.8. In certain embodiments, the mixture of the two or more hydroxypropyl β-cyclodextrin species has a molar substitution value from about 0.8 to 1.0.

In certain embodiments, the mixture of the two or more hydroxypropyl β-cyclodextrin species has an MS value of about 0.5, about 0.53, about 0.56, about 0.59, about 0.62, about 0.65, about 0.68, about 0.71, about 0.74, about 0.77, about 0.8, about 0.83, about 0.86, about 0.89, about 0.92, about 0.95, about 0.98, about 1, about 1.02, about 1.05, about 1.08, about 1.11, about 1.14, about 1.17, or about 1.2.

In certain embodiments, the MS of the mixture of hydroxypropyl β-cyclodextrin species is about 0.40, about 0.41, about 0.42, about 0.43, about 0.44, about 0.45, about 0.46, about 0.47, about 0.48, about 0.49, about 0.50, about 0.51, about 0.52, about 0.53, about 0.54, about 0.55, about 0.56, about 0.57, about 0.58, about 0.59, about 0.60, about 0.61, about 0.62, about 0.63, about 0.64, about 0.65, about 0.66, about 0.69, about 0.68, about 0.69, about 0.70, about 0.71, about 0.72, about 0.73, about 0.74, about 0.75, about 0.76, about 0.77, about 0.78, about 0.79, or about 0.80. In some embodiments, the MS of the mixture of hydroxypropyl β-cyclodextrin species is about 0.59, about 0.60, about 0.61, about 0.62, about 0.63, about 0.64, about 0.65, about 0.66, about 0.69, about 0.68, about 0.69, about 0.70, about 0.71, about 0.72, or about 0.73.

Hydroxypropyl groups can be bonded to the β-cyclodextrins as monomers or can themselves be sequentially bonded to one or more additional hydroxypropyl groups to form hydroxypropyl oligomers which are then bonded to the β-cyclodextrins. In certain embodiments, the hydroxypropyl groups are substituted at the hydroxyl positions of the β-cyclodextrins as hydroxypropyl chains of the structure —[CH₂CH(CH₃)O]ₙH, wherein n≥1 and the average number of hydroxypropyl chains per β-cyclodextrin is from about 3 to about 7. In some embodiments, the average number of hydroxypropyl chains per β-cyclodextrin is from about 3 to about 6, about 3 to about 5, about 3 to about 4, about 4 to about 7, about 4 to about 6, about 4 to about 5, about 5 to about 7, about 5 to about 6, or about 6 to about 7. In some embodiments, n is 1, 2, 3 or 4.

In one illustrative example, a hydroxypropyl chain of the structure-CH₂CH(CH₃)OH includes one hydroxypropyl group in the hydroxypropyl chain (n=1). In another illustrative example a hydroxypropyl chain of the structure —[CH₂CH(CH₃)O]₃H includes three hydroxypropyl groups in the hydroxypropyl chain (n=3).

In certain embodiments, the average number of hydroxypropyl chains per β-cyclodextrin is 3.3±0.3, 3.4±0.3, 3.6±0.3, or 3.8±0.3. In certain embodiments, the average number of hydroxypropyl chains per β-cyclodextrin is 4.0±0.3, 4.2±0.3, 4.4±0.3, 4.6±0.3, or 4.8±0.3. In certain embodiments, the average number of hydroxypropyl chains per β-cyclodextrin is 5.0±0.3, 5.2±0.3, 5.4±0.3, 5.6±0.3, or 5.8±0.3. In certain embodiments, the average number of hydroxypropyl chains per β-cyclodextrin is 6.0±0.3, 6.2±0.3, 6.4±0.3, 6.6±0.3, or 6.7±0.3.

In certain embodiments, the average number of hydroxypropyl chains per β-cyclodextrin is 3.2±0.2, 3.3±0.2, 3.4±0.2, 3.5±0.2, 3.6±0.2, 3.7±0.2, or 3.8±0.2. In certain embodiments, the average number of hydroxypropyl chains per β-cyclodextrin is 4.0±0.2, 4.1±0.2, 4.2±0.2, 4.3±0.2, 4.4±0.2, 4.5±0.2, 4.6±0.2, 4.7±0.2, or 4.8±0.2. In certain embodiments, the average number of hydroxypropyl chains per β-cyclodextrin is 5.0±0.2, 5.1±0.2, 5.2±0.2, 5.3±0.2, 5.4±0.2, 5.5±0.2, 5.6±0.2, 5.7±0.2, or 5.8±0.2. In certain embodiments, the average number of hydroxypropyl chains per β-cyclodextrin is 6.0±0.2, 6.1±0.2, 6.2±0.2, 6.3±0.2, 6.4±0.2, 6.5±0.2, 6.6±0.2, 6.7±0.2, or 6.8±0.2.

In certain embodiments, the average number of hydroxypropyl chains per β-cyclodextrin is 3.1±0.1, 3.2±0.1, 3.3±0.1, 3.4±0.1, 3.5±0.1, 3.6±0.1, 3.7±0.1, 3.8±0.1, or 3.90.1. In certain embodiments, the average number of hydroxypropyl chains per β-cyclodextrin is 4.0±0.1, 4.1±0.1, 4.2±0.1, 4.3±0.1, 4.4±0.1, 4.5±0.1, 4.6±0.1, 4.7±0.1, 4.8±0.1, or 4.9±0.1. In certain embodiments, the average number of hydroxypropyl chains per β-cyclodextrin is 5.0±0.1, 5.1±0.1, 5.2±0.1, 5.3±0.1, 5.4±0.1, 5.5±0.1, 5.6±0.1, 5.7±0.1, 5.8±0.1, or 5.9±0.1. In certain embodiments, the average number of hydroxypropyl chains per β-cyclodextrin is 6.0±0.1, 6.1±0.1, 6.2±0.1, 6.3±0.1, 6.4±0.1, 6.5±0.1, 6.6±0.1, 6.7±0.1, 6.8±0.1, or 6.9±0.1.

In certain embodiments, at least about 50% of the hydroxypropyl chains have n=1. In some embodiments, about 55%, about 60%, about 65%, about 70%, about 75%, about 80%, about 85%, about 90%, about 95%, or about 97%, of the hydroxypropyl chains have n=1. In some embodiments, at least 70% of the hydroxypropyl chains have n=1. In some embodiments, at least 90% of the hydroxypropyl chains have n=1.

In certain embodiments, percentage of the hydroxypropyl chains that have n=1 ranges from about 50% to about 99%, such as about 55% to about 99%, about 60% to about 99%, about 65% to about 99%, about 70% to about 99%, about 75% to about 99%, about 80% to about 99%, about 85% to about 99%, about 90% to about 99%, about 95% to about 99%; such as from about 50% to about 97%, such as about 55% to about 97%, about 60% to about 97%, about 65% to about 97%, about 70% to about 97%, about 75% to about 97%, about 80% to about 97%, about 85% to about 97%, about 90% to about 97%, about 95% to about 97%; such as from about 50% to about 95%, about 55% to about 95%, about 60% to about 95%, about 65% to about 95%, about 70% to about 95%, about 75% to about 95%, about 80% to about 95%, about 85% to about 95%, about 90% to about 95%; such as from about 50% to about 90%, about 55% to about 90%, about 60% to about 90%, about 65% to about 90%, about 70% to about 90%, about 75% to about 90%, about 80% to about 90%, about 85% to about 90%; such as from about 50% to about 85%, about 55% to about 85%, about 60% to about 85%, about 65% to about 85%, about 70% to about 85%, about 75% to about 85%, about 80% to about 85%; such as from about 50% to about 80%, about 55% to about 80%, about 60% to about 80%, about 65% to about 80%, about 70% to about 80%, about 75% to about 80%; such as from about 50% to about 75%, about 55% to about 75%, about 60% to about 75%, about 65% to about 75%, about 70% to about 75%; such as from about 50% to about 70%, about 55% to about 70%, about 60% to about 70%, about 65% to about 70%; such as from about 50% to about 65%, about 55% to about 65%, about 60% to about 65%; such as from about 50% to about 60%, about 55% to about 60%; or such as from about 50% to about 55%.

In certain embodiments, less than about 3%, less than about 5%, less than about 10%, less than about 15%, less than about 20%, less than about 25%, less than about 30%, less than about 35%, less than about 40%, less than about 45%, or less than about 50%, of the hydroxypropyl chains have n=2. In some embodiments, less than about 30% of the hydroxypropyl chains have n=2. In some embodiments, less than 10% of the hydroxypropyl chains have n=2.

In certain embodiments, the percentage of the hydroxypropyl chains that have n=2 ranges from about 5% to about 50%, such as about 10% to about 50%, about 15% to about 50%, about 20% to about 50%, about 25% to about 50%, about 30% to about 50%, about 35% to about 50%, about 40% to about 50%, about 45% to about 50%; such as from about 5% to about 45%, about 10% to about 45%, about 15% to about 45%, about 20% to about 45%, about 25% to about 45%, about 30% to about 45%, about 35% to about 45%, about 40% to about 45%; such as from about 5% to about 40%, about 10% to about 40%, about 15% to about 40%, about 20% to about 40%, about 25% to about 40%, about 30% to about 40%, about 35% to about 40%; such as from about 5% to about 35%, about 10% to about 35%, about 15% to about 35%, about 20% to about 35%, about 25% to about 35%, about 30% to about 35%; such as from about 5% to about 30%, about 10% to about 30%, about 15% to about 30%, about 20% to about 30%, about 25% to about 30%; such as from about 5% to about 25%, about 10% to about 25%, about 15% to about 25%, about 20% to about 25%; such as from about 5% to about 20%, about 10% to about 20%, about 15% to about 20%; such as from about 5% to about 15%, about 10% to about 15%; or about 5% to about 10%.

In some embodiments, less than about 3%, less than about 5%, less than about 10%, less than about 15%, less than about 20%, less than about 25%, less than about 30%, less than about 35%, less than about 40%, less than about 45%, or less than about 50%, of the hydroxypropyl chains have n>2. In some embodiments, less than about 10% of the hydroxypropyl chains have n>2.

In certain embodiments, the percentage of the hydroxypropyl chains that have n>2 ranges from about 5% to about 50%, about 10% to about 50%, about 15% to about 50%, about 20% to about 50%, about 25% to about 50%, about 30% to about 50%, about 35% to about 50%, about 40% to about 50%, about 45% to about 50%, about 5% to about 45%, about 10% to about 45%, about 15% to about 45%, about 20% to about 45%, about 25% to about 45%, about 30% to about 45%, about 35% to about 45%, about 40% to about 45%, about 5% to about 40%, about 10% to about 40%, about 15% to about 40%, about 20% to about 40%, about 25% to about 40%, about 30% to about 40%, about 35% to about 40%, about 5% to about 35%, about 10% to about 35%, about 15% to about 35%, about 20% to about 35%, about 25% to about 35%, about 30% to about 35%, about 5% to about 30%, about 10% to about 30%, about 15% to about 30%, about 20% to about 30%, about 25% to about 30%, about 5% to about 25%, about 10% to about 25%, about 15% to about 25%, about 20% to about 25%, about 5% to about 20%, about 10% to about 20%, about 15% to about 20%, about 5% to about 15%, about 10% to about 15%, or about 5% to about 10%.

In certain embodiments, the average number of hydroxypropyl chains per β-cyclodextrin is from about 4 to about 6. In some embodiments, at least about 60% of the β-cyclodextrins collectively have an average number of hydroxypropyl chains per β-cyclodextrin of from about 4 to about 6. In some embodiments, at least about 65%, at least about 70%, at least about 75%, at least about 80%, at least about 85%, at least about 90%, at least about 95%, or at least about 97%, of the β-cyclodextrins collectively have an average number of hydroxypropyl chains per β-cyclodextrin of from about 4 to about 6. In some embodiments, the percentage of the β-cyclodextrins that collectively have an average number of hydroxypropyl chains per β-cyclodextrin of from about 4 to about 6 ranges from about 60% to about 97%, about 65% to about 97%, about 70% to about 97%, about 75% to about 97%, about 80% to about 97%, about 85% to about 97%, about 90% to about 97%, about 60% to about 95%, about 65% to about 95%, about 70% to about 95%, about 75% to about 95%, about 80% to about 95%, about 85% to about 95%, about 90% to about 95%, about 60% to about 90%, about 65% to about 90%, about 70% to about 90%, about 75% to about 90%, about 80% to about 90%, about 85% to about 90%, about 60% to about 85%, about 65% to about 85%, about 70% to about 85%, about 75% to about 85%, about 80% to about 85%, about 60% to about 80%, about 65% to about 80%, about 70% to about 80%, about 75% to about 80%, about 60% to about 75%, about 65% to about 75%, about 70% to about 75%, about 60% to about 70%, about 65% to about 70%, or about 60% to about 65%.

In certain embodiments, the hydroxypropyl β-cyclodextrin compositions as described herein comprise about 0.05% w/w or less, about 0.06% w/w or less, about 0.07% w/w or less, about 0.08% w/w or less, about 0.09% w/w or less, about 0.1% w/w or less, about 0.15% w/w or less, about 0.2% w/w or less, about 0.3% w/w or less, about 0.4% w/w or less, about 0.5% w/w or less, about 0.6% w/w or less, about 0.7% w/w or less, about 0.8% w/w or less, about 0.9% w/w or less, about 1.0% w/w or less, about 1.1% w/w or less, about 1.2% w/w or less, about 1.3% w/w or less, about 1.4% w/w or less, about 1.5% w/w or less, about 1.6% w/w or less, about 1.7% w/w or less, about 1.8% w/w or less, about 1.9% w/w or less, about 2% w/w or less of unsubstituted β-cyclodextrin. In some embodiments, the hydroxypropyl β-cyclodextrin composition comprises about 0.2% w/w or less of unsubstituted β-cyclodextrin. In some embodiments, the hydroxypropyl β-cyclodextrin composition comprises about 0.15% w/w or less of unsubstituted β-cyclodextrin.

In certain embodiments, the amount of unsubstituted β-cyclodextrin in the hydroxypropyl β-cyclodextrin compositions as described herein is from about 0.05% w/w to about 2% w/w, about 0.05% w/w to about 1.5% w/w, about 0.05% w/w to about 1.4% w/w, about 0.05% w/w to about 1.3% w/w, about 0.05% w/w to about 1.2% w/w, about 0.05% w/w to about 1.1% w/w, about 0.05% w/w to about 1.0% w/w, about 0.05% w/w to about 0.8% w/w, about 0.05% w/w to about 0.6% w/w, about 0.05% w/w to about 0.5% w/w, about 0.05% w/w to about 0.4% w/w, about 0.05% w/w to about 0.3% w/w, about 0.05% w/w to about 0.2% w/w, about 0.05% w/w to about 0.1% w/w, about 0.05% w/w to about 0.07% w/w, about 0.07% w/w to about 1.5% w/w, about 0.07% w/w to about 1.4% w/w, about 0.07% w/w to about 1.3% w/w, about 0.07% w/w to about 1.2% w/w, about 0.07% w/w to about 1.1% w/w, about 0.07% w/w to about 1.0% w/w, about 0.07% w/w to about 0.8% w/w, about 0.07% w/w to about 0.6% w/w, about 0.07% w/w to about 0.5% w/w, about 0.07% w/w to about 0.4% w/w, about 0.07% w/w to about 0.3% w/w, about 0.07% w/w to about 0.2% w/w, about 0.07% w/w to about 0.1% w/w, about 0.1% w/w to about 1.5% w/w, about 0.1% w/w to about 1.4% w/w, about 0.1% w/w to about 1.3% w/w, about 0.1% w/w to about 1.2% w/w, about 0.1% w/w to about 1.1% w/w, about 0.1% w/w to about 1.0% w/w, about 0.1% w/w to about 0.8% w/w, about 0.1% w/w to about 0.6% w/w, about 0.1% w/w to about 0.5% w/w, about 0.1% w/w to about 0.4% w/w, about 0.1% w/w to about 0.3% w/w, about 0.1% w/w to about 0.2% w/w, about 0.2% w/w to about 1.5% w/w, about 0.2% w/w to about 1.4% w/w, about 0.2% w/w to about 1.3% w/w, about 0.2% w/w to about 1.2% w/w, about 0.2% w/w to about 1.1% w/w, about 0.2% w/w to about 1.0% w/w, about 0.2% w/w to about 0.8% w/w, about 0.2% w/w to about 0.6% w/w, about 0.2% w/w to about 0.5% w/w, about 0.2% w/w to about 0.4% w/w, about 0.2% w/w to about 0.3% w/w, about 0.3% w/w to about 1.5% w/w, about 0.3% w/w to about 1.4% w/w, about 0.3% w/w to about 1.3% w/w, about 0.3% w/w to about 1.2% w/w, about 0.3% w/w to about 1.1% w/w, about 0.3% w/w to about 1.0% w/w, about 0.3% w/w to about 0.8% w/w, about 0.3% w/w to about 0.6% w/w, about 0.3% w/w to about 0.5% w/w, about 0.3% w/w to about 0.4% w/w, about 0.4% w/w to about 1.5% w/w, about 0.4% w/w to about 1.4% w/w, about 0.4% w/w to about 1.3% w/w, about 0.4% w/w to about 1.2% w/w, about 0.4% w/w to about 1.1% w/w, about 0.4% w/w to about 1.0% w/w, about 0.4% w/w to about 0.8% w/w, about 0.4% w/w to about 0.6% w/w, about 0.4% w/w to about 0.5% w/w, about 0.5% w/w to about 1.5% w/w, about 0.5% w/w to about 1.4% w/w, about 0.5% w/w to about 1.3% w/w, about 0.5% w/w to about 1.2% w/w, about 0.5% w/w to about 1.1% w/w, about 0.5% w/w to about 1.0% w/w, about 0.5% w/w to about 0.8% w/w, about 0.5% w/w to about 0.6% w/w, about 0.6% w/w to about 1.5% w/w, about 0.6% w/w to about 1.4% w/w, about 0.6% w/w to about 1.3% w/w, about 0.6% w/w to about 1.2% w/w, about 0.6% w/w to about 1.1% w/w, about 0.6% w/w to about 1.0% w/w, about 0.6% w/w to about 0.8% w/w, about 0.8% w/w to about 1.5% w/w, about 0.8% w/w to about 1.4% w/w, about 0.8% w/w to about 1.3% w/w, about 0.8% w/w to about 1.2% w/w, about 0.8% w/w to about 1.1% w/w, about 0.8% w/w to about 1.0% w/w, about 1.0% w/w to about 1.5% w/w, about 1.0% w/w to about 1.4% w/w, about 1.0% w/w to about 1.3% w/w, about 1.0% w/w to about 1.2% w/w, about 1.0% w/w to about 1.1% w/w, about 1.1% w/w to about 1.5% w/w, about 1.1% w/w to about 1.4% w/w, about 1.1% w/w to about 1.3% w/w, about 1.1% w/w to about 1.2% w/w, about 1.2% w/w to about 1.5% w/w, about 1.2% w/w to about 1.4% w/w, about 1.2% w/w to about 1.3% w/w, about 1.3% w/w to about 1.5% w/w, about 1.3% w/w to about 1.4% w/w, or about 1.4% w/w to about 1.5% w/w. In some embodiments, the amount of unsubstituted β-cyclodextrin in the hydroxypropyl β-cyclodextrin compositions as described herein is from about 0.05% w/w to about 2% w/w. In some embodiments, the amount of unsubstituted β-cyclodextrin in the hydroxypropyl β-cyclodextrin compositions as described herein is from about 0.1% w/w to about 0.2% w/w.

In certain embodiments, the hydroxypropyl β-cyclodextrin compositions of the present invention may comprise an impurity resulting from the chemical synthesis of the hydroxypropyl β-cyclodextrin species. In some embodiments, the impurity is propylene glycol.

In certain embodiments, a hydroxypropyl β-cyclodextrin composition described herein comprises about 2.0% or less, about 2.1% w/w or less, about 2.2% w/w or less, about 2.3% w/w or less, about 2.4% w/w or less, about 2.5% w/w or less, about 2.6% w/w or less, about 2.7% w/w or less, about 2.8% w/w or less, about 2.9% w/w or less, or about 3% w/w or less of propylene glycol. In some embodiments, a hydroxypropyl β-cyclodextrin composition described herein comprises about 2.5% w/w or less of propylene glycol.

In certain embodiments, a hydroxypropyl β-cyclodextrin composition described herein comprises 2.0% w/w or less, 2.1% w/w or less, 2.2% w/w or less, 2.3% w/w or less, 2.4% w/w or less, 2.5% w/w or less, 2.6% w/w or less, 2.7% w/w or less, 2.8% w/w or less, 2.9% w/w or less, or 3% w/w or less of propylene glycol. In some embodiments, the hydroxypropyl β-cyclodextrin compositions as described herein comprise 2.5% w/w or less of propylene glycol.

In certain embodiments, the amount of propylene glycol in a hydroxypropyl β-cyclodextrin composition described herein is from about 2.0% w/w to about 3.0% w/w, about 2.0% w/w to about 2.9% w/w, about 2.0% w/w to about 2.8% w/w, about 2.0% w/w to about 2.7% w/w, about 2.0% w/w to about 2.6% w/w, about 2.0% w/w to about 2.5% w/w, about 2.0% w/w to about 2.4% w/w, about 2.0% w/w to about 2.3% w/w, about 2.0% w/w to about 2.2% w/w, about 2.0% w/w to about 2.1% w/w, about 2.1% w/w to about 3.0% w/w, about 2.1% w/w to about 2.9% w/w, about 2.1% w/w to about 2.8% w/w, about 2.1% w/w to about 2.7% w/w, about 2.1% w/w to about 2.6% w/w, about 2.1% w/w to about 2.5% w/w, about 2.1% w/w to about 2.4% w/w, about 2.1% w/w to about 2.3% w/w, about 2.1% w/w to about 2.2% w/w, about 2.2% w/w to about 3.0% w/w, about 2.2% w/w to about 2.9% w/w, about 2.2% w/w to about 2.8% w/w, about 2.2% w/w to about 2.6% w/w, about 2.2% w/w to about 2.5% w/w, about 2.2% w/w to about 2.4% w/w, about 2.2% w/w to about 2.3% w/w, about 2.3% w/w to about 3.0% w/w, about 2.3% w/w to about 2.9% w/w, about 2.3% w/w to about 2.8% w/w, about 2.3% w/w to about 2.7% w/w, about 2.3% w/w to about 2.6% w/w, about 2.3% w/w to about 2.5% w/w, about 2.3% w/w to about 2.4% w/w, about 2.4% w/w to about 3.0% w/w, about 2.4% w/w to about 2.9% w/w, about 2.4% w/w to about 2.8% w/w, about 2.4% w/w to about 2.7% w/w, about 2.4% w/w to about 2.6% w/w, about 2.4% w/w to about 2.5% w/w, about 2.5% w/w to about 3.0% w/w, about 2.5% w/w to about 2.9% w/w, about 2.5% w/w to about 2.8% w/w, about 2.5% w/w to about 2.7% w/w, about 2.5% w/w to about 2.6% w/w, about 2.6% w/w to about 3.0% w/w, about 2.6% w/w to about 2.9% w/w, about 2.6% w/w to about 2.8% w/w, about 2.6% w/w to about 2.7% w/w, about 2.7% w/w to about 3.0% w/w, about 2.7% w/w to about 2.9% w/w, about 2.7% w/w to about 2.8% w/w, about 2.8% w/w to about 3.0% w/w, about 2.8% w/w to about 2.9% w/w, or about 2.9% w/w to about 3.0% w/w. In some embodiments, the amount of propylene glycol in the hydroxypropyl β-cyclodextrin compositions as described herein is from about 2.0% w/w to about 3.0% w/w.

In certain embodiments, the hydroxypropyl β-cyclodextrin composition comprises a mixture of two or more hydroxypropyl β-cyclodextrin species, wherein each of the two or more hydroxypropyl β-cyclodextrin species has a different degree of hydroxypropylation of the β-cyclodextrin ring, and wherein the hydroxypropyl β-cyclodextrin composition comprises 0.15% w/w or less of unsubstituted β-cyclodextrin.

In certain embodiments, the hydroxypropyl β-cyclodextrin composition comprises a mixture of two or more hydroxypropyl β-cyclodextrin species, wherein each of the two or more hydroxypropyl β-cyclodextrin species has a different degree of hydroxypropylation of the β-cyclodextrin ring, wherein the mixture of two or more hydroxypropyl β-cyclodextrin species has a molar substitution value from about 0.59 to about 0.73, and wherein the hydroxypropyl β-cyclodextrin composition comprises 2.5% w/w or less of propylene glycol and 0.15% w/w or less of unsubstituted β-cyclodextrin.

In certain embodiments, the hydroxypropyl β-cyclodextrin composition comprises a mixture of two or more hydroxypropyl β-cyclodextrin species, wherein each of the two or more hydroxypropyl β-cyclodextrin species has a different degree of hydroxypropylation of the β-cyclodextrin ring, wherein the mixture of two or more hydroxypropyl β-cyclodextrin species has a molar substitution value from about 0.59 to about 0.8, and wherein the hydroxypropyl β-cyclodextrin composition comprises 2.5% w/w or less of propylene glycol and 0.15% w/w or less of unsubstituted β-cyclodextrin.

In certain embodiments, the hydroxypropyl β-cyclodextrin composition comprises a mixture of two or more hydroxypropyl β-cyclodextrin species, wherein each of the two or more hydroxypropyl β-cyclodextrin species has a different degree of hydroxypropylation of the β-cyclodextrin ring, wherein the mixture of two or more hydroxypropyl β-cyclodextrin species has a molar substitution value from about 0.8 to about 1.0, and wherein the hydroxypropyl β-cyclodextrin composition comprises 2.5% w/w or less of propylene glycol, preferably less than 0.01% w/w propylene glycol, most preferably an undetectable amount of propylene glycol, and 0.16% w/w or less of unsubstituted β-cyclodextrin.

In certain embodiments, the hydroxypropyl β-cyclodextrin composition comprises a solution comprising about 5% (w/v) to about 40% (w/v), about 10% (w/v) to about 40% (w/v), about 15% (w/v) to about 40% (w/v), about 20% (w/v) to about 40% (w/v), about 25% (w/v) to about 40% (w/v), about 30% (w/v) to about 40% (w/v), about 35% (w/v) to about 40% (w/v), about 5% (w/v) to about 35% (w/v), about 5% (w/v) to about 30% (w/v), about 5% (w/v) to about 25% (w/v), about 5% (w/v) to about 20% (w/v), about 5% (w/v) to about 15% (w/v), about 5% (w/v) to about 10% (w/v), about 10% (w/v) to about 35% (w/v), about 10% (w/v) to about 30% (w/v), about 10% (w/v) to about 25% (w/v), about 10% (w/v) to about 20% (w/v), about 10% (w/v) to about 15% (w/v), about 15% (w/v) to about 35% (w/v), about 15% (w/v) to about 30% (w/v), about 15% (w/v) to about 25% (w/v), about 15% (w/v) to about 20% (w/v), about 20% (w/v) to about 35% (w/v), about 20% (w/v) to about 30% (w/v), about 20% (w/v) to about 25% (w/v), about 25% (w/v) to about 35% (w/v), about 25% (w/v) to about 30% (w/v), or about 30% (w/v) to about 35% (w/v) of one or more hydroxypropyl β-cyclodextrin species.

In certain embodiments, the hydroxypropyl β-cyclodextrin composition comprises a 5% (w/v), 10% (w/v), 15% (w/v), 20% (w/v), 25% (w/v), 30% (w/v), 35% (w/v) or 40% (w/v) solution of one or more hydroxypropyl β-cyclodextrin species. In some embodiments, the hydroxypropyl β-cyclodextrin composition comprises a 25% (w/v) solution of one or more hydroxypropyl β-cyclodextrin species. In some embodiments, the 25% (w/v) solution of one or more hydroxypropyl β-cyclodextrin species is a 25% (w/v) aqueous solution of one or more hydroxypropyl β-cyclodextrin species.

In certain embodiments, the hydroxypropyl β-cyclodextrin composition comprises the Trappsol® Cyclo™ hydroxypropyl β-cyclodextrin composition, available from Cyclo Therapeutics, Inc.

Treatment of Early Alzheimer's Disease with Hydroxypropyl β-Cyclodextrins

### Effect of Cholesterol on Early Alzheimer's Disease

Early Alzheimer's disease neuropathology may be characterized by 1) amyloid-beta (Aβ) containing plaques, 2) neurofibrillary tangles composed of neurofilaments, or 3) hyperphosphorylated tau protein. Other critical components include LE/LY abnormalities, cholesterol accumulation, APP processing inflammation, and apoptosis. While the exact roles of the Aβ plaques and tau tangles in Alzheimer's disease are unknown, it is believed that they play a critical role in blocking communication among nerve cells and disrupting the critical processes that cells require in order to survive. Moreover, studies have demonstrated that Aβ is neurotoxic and there is evidence to suggest that it appears to be responsible for initiating the memory loss associated with Alzheimer's disease.

Cholesterol is considered to be essential for cell structure, function and signaling. Approximately twenty-three percent of all cholesterol in the body is located within the brain; with neurons and astrocytes containing the largest amounts. Cholesterol is not uniformly distributed: as variations exist both within each particular membrane and across different membranes of the same cell. Within each membrane, cholesterol is concentrated at nano/micro domains termed ‘lipid rafts.’ In neurons, these rafts are highly dynamic, thought to be a result of their high metabolic demands and requirement for plasticity and re-modelling throughout life. Rafts have also been detected at neuronal synapses, where they contribute to pre- and post-synaptic function. Within a single neuron, the relative distribution of cholesterol also varies across intracellular membranes. The vast majority (>90%) of cholesterol is located within the plasma membrane. Once across the plasma membrane, most cholesterol is shuttled to endosomes/lysosomes and then on to certain organelles for processing, where it may be incorporated into other organelle membranes (i.e., mitochondria, lysosome, endoplasmic reticulum) or esterified and stored in the form of cytosolic lipid droplets.

Subjects suffering from Alzheimer's disease (e.g., early Alzheimer's disease) often suffer from cholesterol imbalances and existing studies suggest that these imbalances may be responsible for Aβ and tau accumulation. Furthermore, neurons, due to their high metabolic demands, may experience an increased level of oxidative stress. Oxidative stress has been linked to abnormal cholesterol accumulation and processing. Studies have shown that that young or juvenile neurons have lower membrane cholesterol levels than mature neurons. Furthermore, cadaver studies of Alzheimer's disease patients have also shown that levels of cellular cholesterol are significantly increased in the membranes within vulnerable brain regions, but not non-vulnerable brain regions. The amount of cholesterol in these membranes has also been found to be greater in patients with more severe cognitive symptoms than in patients with mild impairment.

Cells of the brain are largely cut off from blood cholesterol supply because the blood-brain barrier prevents entry of cholesterol rich lipoproteins. Therefore, most CNS cholesterol is made locally. On the other hand, there is a constant efflux of cholesterol from the brain through neuron specific enzyme Cytochrome 46A1. This enzyme hydroxylates cholesterol to 24S-hydroxycholesterol (24-OHC), which crosses the blood brain barrier, to enter circulation. Altered CYP46A1 expression has been associated with several neurodegenerative diseases and changes in cognition. A second mechanism of efflux is HDL, which can cross the blood-brain barrier, and transports cholesterol out of the brain, to the plasma and then on to the liver for eventual removal. Low and/or dysfunctional HDL particles are known risk factors for developing AD. Finally, apolipoprotein E (APOE) isoform type 84, which is a form of LDL and regulates brain cholesterol metabolism and transport may be the strongest identified genetic risk factor for the development of AD.

There is an established link between Aβ and cholesterol, as both the generation and clearance of Aβ are regulated by cholesterol. Increasing cholesterol content, particularly in domains such as the plasma membrane, can result in increased Aβ levels, as has been shown in both cell culture and most animal models of AD. Tau toxicity may also depend on cellular cholesterol levels. High cholesterol diets have been shown to increase tau hyper phosphorylation. Studies have shown that as cholesterol concentration increases, so does the susceptibility of neurons to Aβ-dependent calpain activation. Calpain activation is known to cleave tau and generate toxic fragments. These cleaved tau forms can induce neuronal death, synapse loss, and/or behavioral deficits. Young neurons which have measurably less cholesterol than aged neurons, also contain less phosphorylated tau (p-tau).

Treating AD with Hydroxypropyl β-Cyclodextrins

In one aspect, provided herein is a method of treating early Alzheimer's disease in a human patient in need thereof, the method generally comprising administering to the human patient an effective amount of a hydroxypropyl β-cyclodextrin composition disclosed herein.

In certain embodiments, the early Alzheimer's disease is Alzheimer's disease with mild cognitive impairment or mild Alzheimer's disease. In certain embodiments, the early Alzheimer's disease is Alzheimer's disease with mild cognitive impairment. In certain embodiments, the early Alzheimer's disease is mild Alzheimer's disease.

In some embodiments, at the time of initiation of administration of the effective amount of the hydroxypropyl β-cyclodextrin composition, the human patient exhibits progressive cognitive decline.

In some embodiments, at the time of initiation of administration of the effective amount of the hydroxypropyl β-cyclodextrin composition, the human patient has exhibited progressive cognitive decline for at least about 6 months, about 7 months, about 8 months, about 9 months, about 10 months, about 11 months, about 1 year, about 1 year and 1 month, about 1 year and 2 months, about 1 year and 3 months, about 1 year and 4 months, about 1 year and 5 months, or about 1 year and 6 months. In some embodiments, at the time of initiation of administration of the effective amount of the hydroxypropyl β-cyclodextrin composition, the human patient has exhibited progressive cognitive decline for at least about 1 year.

In some embodiments, at the time of initiation of administration of the effective amount of the hydroxypropyl β-cyclodextrin composition, the human patient has exhibited progressive cognitive decline for about 6 months, about 7 months, about 8 months, about 9 months, about 10 months, about 11 months, about 1 year, about 1 year and 1 month, about 1 year and 2 months, about 1 year and 3 months, about 1 year and 4 months, about 1 year and 5 months, or about 1 year and 6 months.

In certain embodiments, at the time of initiation of administration of the effective amount of the hydroxypropyl β-cyclodextrin composition, the human patient has a global Clinical Dementia Rating (CDR) scale score of about 0.1, about 0.2, about 0.3, about 0.4, about 0.5, about 0.6, about 0.7, about 0.8, about 0.9, or about 1.0.

In certain embodiments, at the time of initiation of administration of the effective amount of the hydroxypropyl β-cyclodextrin composition, the human patient has a global Clinical Dementia Rating (CDR) scale score of between about 0.1 and about 0.8, about 0.2 and about 0.8, about 0.3 and about 0.8, about 0.4 and about 0.8, about 0.5 and about 0.8, about 0.6 and about 0.8, about 0.7 and about 0.8, about 0.1 and about 0.9, about 0.2 and about 0.9, about 0.3 and about 0.9, about 0.4 and about 0.9, about 0.5 and about 0.9, about 0.6 and about 0.9, about 0.7 and about 0.9, about 0.8 and about 0.9, about 0.1 and about 1.0, about 0.2 and about 1.0, about 0.3 and about 1.0, about 0.4 and about 1.0, about 0.5 and about 1.0, about 0.6 and about 1.0, about 0.7 and about 1.0, about 0.8 and about 1.0, about 0.9 and about 1.0, about 0.1 and about 1.1, about 0.2 and about 1.1, about 0.3 and about 1.1, about 0.4 and about 1.1, about 0.5 and about 1.1, about 0.6 and about 1.1, about 0.7 and about 1.1, about 0.8 and about 1.1, about 0.9 and about 1.1, about 1.0 and about 1.1, about 0.1 and about 1.2, about 0.2 and about 1.2, about 0.3 and about 1.2, about 0.4 and about 1.2, about 0.5 and about 1.2, about 0.6 and about 1.2, about 0.7 and about 1.2, about 0.8 and about 1.2, about 0.9 and about 1.2, about 1.0 and about 1.2, or about 1.1 and about 1.2. In certain embodiments, at the time of initiation of administration of the effective amount of the hydroxypropyl β-cyclodextrin composition, the human patient has a global Clinical Dementia Rating (CDR) scale score of between about 0.5 and about 1.

In certain embodiments, at the time of initiation of administration of the effective amount of the hydroxypropyl β-cyclodextrin composition, the human patient has a CDR Memory Box score of about 0.1 or greater, about 0.2 or greater, about 0.3 or greater, about 0.4 or greater, about 0.5 or greater, about 0.6 or greater, about 0.7 or greater, about 0.8 or greater, about 0.9 or greater, or about 1.0 or greater. In certain embodiments, at the time of initiation of administration of the effective amount of the hydroxypropyl β-cyclodextrin composition, the human patient has a CDR Memory Box score of about 0.5 or greater.

In certain embodiments, at the time of initiation of administration of the effective amount of the hydroxypropyl β-cyclodextrin composition, the human patient has a global Clinical Dementia Rating (CDR) scale score of about 0.1, about 0.2, about 0.3, about 0.4, about 0.5, about 0.6, about 0.7, about 0.8, about 0.9, or about 1.0, and a CDR Memory Box score of about 0.1 or greater, about 0.2 or greater, about 0.3 or greater, about 0.4 or greater, about 0.5 or greater, about 0.6 or greater, about 0.7 or greater, about 0.8 or greater, about 0.9 or greater, or about 1.0 or greater.

In certain embodiments, at the time of initiation of administration of the effective amount of the hydroxypropyl β-cyclodextrin composition, the human patient has a global Clinical Dementia Rating (CDR) scale score of between about 0.1 and about 0.8, about 0.2 and about 0.8, about 0.3 and about 0.8, about 0.4 and about 0.8, about 0.5 and about 0.8, about 0.6 and about 0.8, about 0.7 and about 0.8, about 0.1 and about 0.9, about 0.2 and about 0.9, about 0.3 and about 0.9, about 0.4 and about 0.9, about 0.5 and about 0.9, about 0.6 and about 0.9, about 0.7 and about 0.9, about 0.8 and about 0.9, about 0.1 and about 1.0, about 0.2 and about 1.0, about 0.3 and about 1.0, about 0.4 and about 1.0, about 0.5 and about 1.0, about 0.6 and about 1.0, about 0.7 and about 1.0, about 0.8 and about 1.0, about 0.9 and about 1.0, about 0.1 and about 1.1, about 0.2 and about 1.1, about 0.3 and about 1.1, about 0.4 and about 1.1, about 0.5 and about 1.1, about 0.6 and about 1.1, about 0.7 and about 1.1, about 0.8 and about 1.1, about 0.9 and about 1.1, about 1.0 and about 1.1, about 0.1 and about 1.2, about 0.2 and about 1.2, about 0.3 and about 1.2, about 0.4 and about 1.2, about 0.5 and about 1.2, about 0.6 and about 1.2, about 0.7 and about 1.2, about 0.8 and about 1.2, about 0.9 and about 1.2, about 1.0 and about 1.2, or about 1.1 and about 1.2, and a CDR Memory Box score of about 0.1 or greater, about 0.2 or greater, about 0.3 or greater, about 0.4 or greater, about 0.5 or greater, about 0.6 or greater, about 0.7 or greater, about 0.8 or greater, about 0.9 or greater, or about 1.0 or greater. In certain embodiments, at the time of initiation of administration of the effective amount of the hydroxypropyl β-cyclodextrin composition the human patient has a global Clinical Dementia Rating (CDR) scale score of between about 0.5 and about 1.0 and a CDR Memory Box score of about 0.5 or greater.

In certain embodiments, at the time of initiation of administration of the effective amount of the hydroxypropyl β-cyclodextrin composition, the human patient exhibits cerebral amyloid-beta (Aβ) pathology.

In certain embodiments, at the time of initiation of administration of the effective amount of the hydroxypropyl β-cyclodextrin composition, the human patient has a Mini-Mental State Examination-2: Standard Version (MMSE-2:SV) score of between about 16 and about 25, about 17 and about 25, about 18 and about 25, about 19 and about 25, about 20 and about 25, about 21 and about 25, about 22 and about 25, about 23 and about 25, about 24 and about 25, about 16 and about 26, about 17 and about 26, about 18 and about 26, about 19 and about 26, about 20 and about 26, about 21 and about 26, about 22 and about 26, about 23 and about 26, about 24 and about 26, about 16 and about 27, about 17 and about 27, about 18 and about 27, about 19 and about 27, about 20 and about 27, about 21 and about 27, about 22 and about 27, about 23 and about 27, about 24 and about 27, about 16 and about 28, about 17 and about 28, about 18 and about 28, about 19 and about 28, about 20 and about 28, about 21 and about 28, about 22 and about 28, about 23 and about 28, about 24 and about 28, about 16 and about 29, about 17 and about 29, about 18 and about 29, about 19 and about 29, about 20 and about 29, about 21 and about 29, about 22 and about 29, about 23 and about 29, about 24 and about 29, about 16 and about 30, about 17 and about 30, about 18 and about 30, about 19 and about 30, about 20 and about 30, about 21 and about 30, about 22 and about 30, about 23 and about 30, about 24 and about 30, about 16 and about 31, about 17 and about 31, about 18 and about 31, about 19 and about 31, about 20 and about 31, about 21 and about 31, about 22 and about 31, about 23 and about 31, or about 24 and about 31. In certain embodiments, at the time of initiation of administration of the effective amount of the hydroxypropyl β-cyclodextrin composition the human patient has a Mini-Mental State Examination-2: Standard Version (MMSE-2:SV) score of between about 20 and about 28.

In certain embodiments, the human patient has previously been administered a pro-cognitive drug and/or a symptomatic therapy for early Alzheimer's disease.

In certain embodiments, the human patient has previously been administered an acetylcholinesterase inhibitor and/or memantine and/or a combination medication of Namenda and Aricept (marketed as Namzaric).

In certain embodiments, the human patient is at least 30 years old, at least 40 years old, at least 50 years old, at least 60 years old, at least 70 years old, at least 80 years old, or at least 90 years old. In certain embodiments, the human patient is at least 30 years old. In certain embodiments, the human patient is at least 40 years old. In certain embodiments, the human patient is at least 50 years old. In certain embodiments, the human patient is at least 60 years old. In certain embodiments, the human patient is at least 70 years old. In certain embodiments, the human patient is at least 80 years old.

In certain embodiments, administering an effective amount of the hydroxypropyl β-cyclodextrin composition comprises administering to the human patient about 100 mg/kg to about 1000 mg/kg, about 200 mg/kg to about 1000 mg/kg, about 300 mg/kg to about 1000 mg/kg, about 400 mg/kg to about 1000 mg/kg, about 500 mg/kg to about 1000 mg/kg, about 600 mg/kg to about 1000 mg/kg, about 700 mg/kg to about 1000 mg/kg, about 750 mg/kg to about 1000 mg/kg, about 800 mg/kg to about 1000 mg/kg, about 900 mg/kg to about 1000 mg/kg, 100 mg/kg to about 2000 mg/kg, about 200 mg/kg to about 2000 mg/kg, about 300 mg/kg to about 2000 mg/kg, about 400 mg/kg to about 2000 mg/kg, about 500 mg/kg to about 2000 mg/kg, about 600 mg/kg to about 2000 mg/kg, about 700 mg/kg to about 2000 mg/kg, about 750 mg/kg to about 2000 mg/kg, about 800 mg/kg to about 2000 mg/kg, about 900 mg/kg to about 2000 mg/kg, about 1000 mg/kg to about 2000 mg/kg, about 1100 mg/kg to about 2000 mg/kg, about 1200 mg/kg to about 2000 mg/kg, about 1250 mg/kg to about 2000 mg/kg, about 1300 mg/kg to about 2000 mg/kg, about 1400 mg/kg to about 2000 mg/kg, about 1500 mg/kg to about 2000 mg/kg, about 1600 mg/kg to about 2000 mg/kg, about 1700 mg/kg to about 2000 mg/kg, about 1800 mg/kg to about 2000 mg/kg, about 1900 mg/kg to about 2000 mg/kg, about 100 mg/kg to about 2100 mg/kg, about 200 mg/kg to about 2100 mg/kg, about 300 mg/kg to about 2100 mg/kg, about 400 mg/kg to about 2100 mg/kg, about 500 mg/kg to about 2100 mg/kg, about 600 mg/kg to about 2100 mg/kg, about 700 mg/kg to about 2100 mg/kg, about 750 mg/kg to about 2100 mg/kg, about 800 mg/kg to about 2100 mg/kg, about 900 mg/kg to about 2100 mg/kg, about 1000 mg/kg to about 2100 mg/kg, about 1100 mg/kg to about 2100 mg/kg, about 1200 mg/kg to about 2100 mg/kg, about 1250 mg/kg to about 2100 mg/kg, about 1300 mg/kg to about 2100 mg/kg, about 1400 mg/kg to about 2100 mg/kg, about 1500 mg/kg to about 2100 mg/kg, about 1600 mg/kg to about 2100 mg/kg, about 1700 mg/kg to about 2100 mg/kg, about 1800 mg/kg to about 2100 mg/kg, about 1900 mg/kg to about 2100 mg/kg, about 2000 mg/kg to about 2100 mg/kg, about 100 mg/kg to about 2500 mg/kg, about 200 mg/kg to about 2500 mg/kg, about 300 mg/kg to about 2500 mg/kg, about 400 mg/kg to about 2500 mg/kg, about 500 mg/kg to about 2500 mg/kg, about 600 mg/kg to about 2500 mg/kg, about 700 mg/kg to about 2500 mg/kg, about 750 mg/kg to about 2500 mg/kg, about 800 mg/kg to about 2500 mg/kg, about 900 mg/kg to about 2500 mg/kg, about 1000 mg/kg to about 2500 mg/kg, about 1100 mg/kg to about 2500 mg/kg, about 1200 mg/kg to about 2500 mg/kg, about 1250 mg/kg to about 2500 mg/kg, about 1300 mg/kg to about 2500 mg/kg, about 1400 mg/kg to about 2500 mg/kg, about 1500 mg/kg to about 2500 mg/kg, about 1600 mg/kg to about 2500 mg/kg, about 1700 mg/kg to about 2500 mg/kg, about 1800 mg/kg to about 2500 mg/kg, about 1900 mg/kg to about 2500 mg/kg, about 2000 mg/kg to about 2500 mg/kg, or about 2100 mg/kg to about 2500 mg/kg of the hydroxypropyl β-cyclodextrin composition. In certain embodiments, administering an effective amount of the hydroxypropyl β-cyclodextrin composition comprises administering to the human patient about 500 mg/kg to about 2000 mg/kg of the hydroxypropyl β-cyclodextrin composition. In certain embodiments, administering an effective amount of the hydroxypropyl β-cyclodextrin composition comprises administering to the human patient about 500 mg/kg to about 1000 mg/kg of the hydroxypropyl β-cyclodextrin composition.

In certain embodiments, administering an effective amount of the hydroxypropyl β-cyclodextrin composition comprises administering to the human patient by intravenous infusion about 100 mg/kg to about 1000 mg/kg, about 200 mg/kg to about 1000 mg/kg, about 300 mg/kg to about 1000 mg/kg, about 400 mg/kg to about 1000 mg/kg, about 500 mg/kg to about 1000 mg/kg, about 600 mg/kg to about 1000 mg/kg, about 700 mg/kg to about 1000 mg/kg, about 750 mg/kg to about 1000 mg/kg, about 800 mg/kg to about 1000 mg/kg, about 900 mg/kg to about 1000 mg/kg, 100 mg/kg to about 2000 mg/kg, about 200 mg/kg to about 2000 mg/kg, about 300 mg/kg to about 2000 mg/kg, about 400 mg/kg to about 2000 mg/kg, about 500 mg/kg to about 2000 mg/kg, about 600 mg/kg to about 2000 mg/kg, about 700 mg/kg to about 2000 mg/kg, about 750 mg/kg to about 2000 mg/kg, about 800 mg/kg to about 2000 mg/kg, about 900 mg/kg to about 2000 mg/kg, about 1000 mg/kg to about 2000 mg/kg, about 1100 mg/kg to about 2000 mg/kg, about 1200 mg/kg to about 2000 mg/kg, about 1250 mg/kg to about 2000 mg/kg, about 1300 mg/kg to about 2000 mg/kg, about 1400 mg/kg to about 2000 mg/kg, about 1500 mg/kg to about 2000 mg/kg, about 1600 mg/kg to about 2000 mg/kg, about 1700 mg/kg to about 2000 mg/kg, about 1800 mg/kg to about 2000 mg/kg, about 1900 mg/kg to about 2000 mg/kg, about 100 mg/kg to about 2100 mg/kg, about 200 mg/kg to about 2100 mg/kg, about 300 mg/kg to about 2100 mg/kg, about 400 mg/kg to about 2100 mg/kg, about 500 mg/kg to about 2100 mg/kg, about 600 mg/kg to about 2100 mg/kg, about 700 mg/kg to about 2100 mg/kg, about 750 mg/kg to about 2100 mg/kg, about 800 mg/kg to about 2100 mg/kg, about 900 mg/kg to about 2100 mg/kg, about 1000 mg/kg to about 2100 mg/kg, about 1100 mg/kg to about 2100 mg/kg, about 1200 mg/kg to about 2100 mg/kg, about 1250 mg/kg to about 2100 mg/kg, about 1300 mg/kg to about 2100 mg/kg, about 1400 mg/kg to about 2100 mg/kg, about 1500 mg/kg to about 2100 mg/kg, about 1600 mg/kg to about 2100 mg/kg, about 1700 mg/kg to about 2100 mg/kg, about 1800 mg/kg to about 2100 mg/kg, about 1900 mg/kg to about 2100 mg/kg, about 2000 mg/kg to about 2100 mg/kg, about 100 mg/kg to about 2500 mg/kg, about 200 mg/kg to about 2500 mg/kg, about 300 mg/kg to about 2500 mg/kg, about 400 mg/kg to about 2500 mg/kg, about 500 mg/kg to about 2500 mg/kg, about 600 mg/kg to about 2500 mg/kg, about 700 mg/kg to about 2500 mg/kg, about 750 mg/kg to about 2500 mg/kg, about 800 mg/kg to about 2500 mg/kg, about 900 mg/kg to about 2500 mg/kg, about 1000 mg/kg to about 2500 mg/kg, about 1100 mg/kg to about 2500 mg/kg, about 1200 mg/kg to about 2500 mg/kg, about 1250 mg/kg to about 2500 mg/kg, about 1300 mg/kg to about 2500 mg/kg, about 1400 mg/kg to about 2500 mg/kg, about 1500 mg/kg to about 2500 mg/kg, about 1600 mg/kg to about 2500 mg/kg, about 1700 mg/kg to about 2500 mg/kg, about 1800 mg/kg to about 2500 mg/kg, about 1900 mg/kg to about 2500 mg/kg, about 2000 mg/kg to about 2500 mg/kg, or about 2100 mg/kg to about 2500 mg/kg of the hydroxypropyl β-cyclodextrin composition. In certain embodiments, administering an effective amount of the hydroxypropyl β-cyclodextrin composition comprises administering to the human patient by intravenous infusion about 500 mg/kg to about 2000 mg/kg of the hydroxypropyl β-cyclodextrin composition. In certain embodiments, administering an effective amount of the hydroxypropyl β-cyclodextrin composition comprises administering to the human patient by intravenous infusion about 500 mg/kg to about 1000 mg/kg of the hydroxypropyl β-cyclodextrin composition.

In certain embodiments, the effective amount of the hydroxypropyl β-cyclodextrin composition is administered to the human patient every 20 days, 21 days, 22 days, 23 days, 24 days, 25 days, 26 days, 27 days, 28 days, 29 days, 30 days, 31 days, 32 days, 33 days, 34 days, 35 days, or 36 days. In certain embodiments, the effective amount of the hydroxypropyl-cyclodextrin composition is administered to the human patient every 28 days.

In certain embodiments, administering an effective amount of the hydroxypropyl β-cyclodextrin composition comprises administering to the human patient by intravenous infusion about 100 mg/kg to about 1000 mg/kg, about 200 mg/kg to about 1000 mg/kg, about 300 mg/kg to about 1000 mg/kg, about 400 mg/kg to about 1000 mg/kg, about 500 mg/kg to about 1000 mg/kg, about 600 mg/kg to about 1000 mg/kg, about 700 mg/kg to about 1000 mg/kg, about 750 mg/kg to about 1000 mg/kg, about 800 mg/kg to about 1000 mg/kg, about 900 mg/kg to about 1000 mg/kg, 100 mg/kg to about 2000 mg/kg, about 200 mg/kg to about 2000 mg/kg, about 300 mg/kg to about 2000 mg/kg, about 400 mg/kg to about 2000 mg/kg, about 500 mg/kg to about 2000 mg/kg, about 600 mg/kg to about 2000 mg/kg, about 700 mg/kg to about 2000 mg/kg, about 750 mg/kg to about 2000 mg/kg, about 800 mg/kg to about 2000 mg/kg, about 900 mg/kg to about 2000 mg/kg, about 1000 mg/kg to about 2000 mg/kg, about 1100 mg/kg to about 2000 mg/kg, about 1200 mg/kg to about 2000 mg/kg, about 1250 mg/kg to about 2000 mg/kg, about 1300 mg/kg to about 2000 mg/kg, about 1400 mg/kg to about 2000 mg/kg, about 1500 mg/kg to about 2000 mg/kg, about 1600 mg/kg to about 2000 mg/kg, about 1700 mg/kg to about 2000 mg/kg, about 1800 mg/kg to about 2000 mg/kg, about 1900 mg/kg to about 2000 mg/kg, about 100 mg/kg to about 2100 mg/kg, about 200 mg/kg to about 2100 mg/kg, about 300 mg/kg to about 2100 mg/kg, about 400 mg/kg to about 2100 mg/kg, about 500 mg/kg to about 2100 mg/kg, about 600 mg/kg to about 2100 mg/kg, about 700 mg/kg to about 2100 mg/kg, about 750 mg/kg to about 2100 mg/kg, about 800 mg/kg to about 2100 mg/kg, about 900 mg/kg to about 2100 mg/kg, about 1000 mg/kg to about 2100 mg/kg, about 1100 mg/kg to about 2100 mg/kg, about 1200 mg/kg to about 2100 mg/kg, about 1250 mg/kg to about 2100 mg/kg, about 1300 mg/kg to about 2100 mg/kg, about 1400 mg/kg to about 2100 mg/kg, about 1500 mg/kg to about 2100 mg/kg, about 1600 mg/kg to about 2100 mg/kg, about 1700 mg/kg to about 2100 mg/kg, about 1800 mg/kg to about 2100 mg/kg, about 1900 mg/kg to about 2100 mg/kg, about 2000 mg/kg to about 2100 mg/kg, about 100 mg/kg to about 2500 mg/kg, about 200 mg/kg to about 2500 mg/kg, about 300 mg/kg to about 2500 mg/kg, about 400 mg/kg to about 2500 mg/kg, about 500 mg/kg to about 2500 mg/kg, about 600 mg/kg to about 2500 mg/kg, about 700 mg/kg to about 2500 mg/kg, about 750 mg/kg to about 2500 mg/kg, about 800 mg/kg to about 2500 mg/kg, about 900 mg/kg to about 2500 mg/kg, about 1000 mg/kg to about 2500 mg/kg, about 1100 mg/kg to about 2500 mg/kg, about 1200 mg/kg to about 2500 mg/kg, about 1250 mg/kg to about 2500 mg/kg, about 1300 mg/kg to about 2500 mg/kg, about 1400 mg/kg to about 2500 mg/kg, about 1500 mg/kg to about 2500 mg/kg, about 1600 mg/kg to about 2500 mg/kg, about 1700 mg/kg to about 2500 mg/kg, about 1800 mg/kg to about 2500 mg/kg, about 1900 mg/kg to about 2500 mg/kg, about 2000 mg/kg to about 2500 mg/kg, or about 2100 mg/kg to about 2500 mg/kg of the hydroxypropyl β-cyclodextrin composition every 20 days, 21 days, 22 days, 23 days, 24 days, 25 days, 26 days, 27 days 28 days, 29 days, 30 days, 31 days, 32 days, 33 days, 34 days, 35 days, or 36 days.

In certain embodiments, administering an effective amount of the hydroxypropyl β-cyclodextrin composition comprises administering to the human patient by intravenous infusion about 500 mg/kg to about 2000 mg/kg of the hydroxypropyl β-cyclodextrin composition, every 28 days. In certain embodiments, administering an effective amount of the hydroxypropyl β-cyclodextrin composition comprises administering to the human patient by intravenous infusion about 500 mg/kg to about 1000 mg/kg of the hydroxypropyl β-cyclodextrin composition, every 28 days.

In certain embodiments, the effective amount of the hydroxypropyl β-cyclodextrin composition is administered to the human patient at regular intervals (e.g., every 21 days, 22 days, 23 days, 24 days, 25 days, 26 days, 27 days, 28 days, 29 days, 30 days, 31 days, 32 days, 33 days, 34 days, or 35 days) for a period of at least about 20 weeks, at least about 21 weeks, at least about 22 weeks, at least about 23 weeks, at least about 24 weeks, at least about 25 weeks, at least about 26 weeks, at least about 27 weeks, or at least about 28 weeks. In certain embodiments, the effective amount of the hydroxypropyl β-cyclodextrin composition is administered to the human patient at regular intervals (e.g., every 21 days, 22 days, 23 days, 24 days, 25 days, 26 days, 27 days, 28 days, 29 days, 30 days, 31 days, 32 days, 33 days, 34 days, or 35 days) for the duration of the human patient's life span.

In certain embodiments, administering an effective amount of the hydroxypropyl β-cyclodextrin composition comprises administering to the human patient by intravenous infusion about 100 mg/kg to about 1000 mg/kg, about 200 mg/kg to about 1000 mg/kg, about 300 mg/kg to about 1000 mg/kg, about 400 mg/kg to about 1000 mg/kg, about 500 mg/kg to about 1000 mg/kg, about 600 mg/kg to about 1000 mg/kg, about 700 mg/kg to about 1000 mg/kg, about 750 mg/kg to about 1000 mg/kg, about 800 mg/kg to about 1000 mg/kg, about 900 mg/kg to about 1000 mg/kg, 100 mg/kg to about 2000 mg/kg, about 200 mg/kg to about 2000 mg/kg, about 300 mg/kg to about 2000 mg/kg, about 400 mg/kg to about 2000 mg/kg, about 500 mg/kg to about 2000 mg/kg, about 600 mg/kg to about 2000 mg/kg, about 700 mg/kg to about 2000 mg/kg, about 750 mg/kg to about 2000 mg/kg, about 800 mg/kg to about 2000 mg/kg, about 900 mg/kg to about 2000 mg/kg, about 1000 mg/kg to about 2000 mg/kg, about 1100 mg/kg to about 2000 mg/kg, about 1200 mg/kg to about 2000 mg/kg, about 1250 mg/kg to about 2000 mg/kg, about 1300 mg/kg to about 2000 mg/kg, about 1400 mg/kg to about 2000 mg/kg, about 1500 mg/kg to about 2000 mg/kg, about 1600 mg/kg to about 2000 mg/kg, about 1700 mg/kg to about 2000 mg/kg, about 1800 mg/kg to about 2000 mg/kg, about 1900 mg/kg to about 2000 mg/kg, about 100 mg/kg to about 2100 mg/kg, about 200 mg/kg to about 2100 mg/kg, about 300 mg/kg to about 2100 mg/kg, about 400 mg/kg to about 2100 mg/kg, about 500 mg/kg to about 2100 mg/kg, about 600 mg/kg to about 2100 mg/kg, about 700 mg/kg to about 2100 mg/kg, about 750 mg/kg to about 2100 mg/kg, about 800 mg/kg to about 2100 mg/kg, about 900 mg/kg to about 2100 mg/kg, about 1000 mg/kg to about 2100 mg/kg, about 1100 mg/kg to about 2100 mg/kg, about 1200 mg/kg to about 2100 mg/kg, about 1250 mg/kg to about 2100 mg/kg, about 1300 mg/kg to about 2100 mg/kg, about 1400 mg/kg to about 2100 mg/kg, about 1500 mg/kg to about 2100 mg/kg, about 1600 mg/kg to about 2100 mg/kg, about 1700 mg/kg to about 2100 mg/kg, about 1800 mg/kg to about 2100 mg/kg, about 1900 mg/kg to about 2100 mg/kg, about 2000 mg/kg to about 2100 mg/kg, about 100 mg/kg to about 2500 mg/kg, about 200 mg/kg to about 2500 mg/kg, about 300 mg/kg to about 2500 mg/kg, about 400 mg/kg to about 2500 mg/kg, about 500 mg/kg to about 2500 mg/kg, about 600 mg/kg to about 2500 mg/kg, about 700 mg/kg to about 2500 mg/kg, about 750 mg/kg to about 2500 mg/kg, about 800 mg/kg to about 2500 mg/kg, about 900 mg/kg to about 2500 mg/kg, about 1000 mg/kg to about 2500 mg/kg, about 1100 mg/kg to about 2500 mg/kg, about 1200 mg/kg to about 2500 mg/kg, about 1250 mg/kg to about 2500 mg/kg, about 1300 mg/kg to about 2500 mg/kg, about 1400 mg/kg to about 2500 mg/kg, about 1500 mg/kg to about 2500 mg/kg, about 1600 mg/kg to about 2500 mg/kg, about 1700 mg/kg to about 2500 mg/kg, about 1800 mg/kg to about 2500 mg/kg, about 1900 mg/kg to about 2500 mg/kg, about 2000 mg/kg to about 2500 mg/kg, or about 2100 mg/kg to about 2500 mg/kg of the hydroxypropyl β-cyclodextrin composition every 20 days, 21 days, 22 days, 23 days, 24 days, 25 days, 26 days, 27 days 28 days, 29 days, 30 days, 31 days, 32 days, 33 days, 34 days, 35 days, or 36 days, for a period of at least about 20 weeks, at least about 21 weeks, at least about 22 weeks, at least about 23 weeks, at least about 24 weeks, at least about 25 weeks, at least about 26 weeks, at least about 27 weeks, or at least about 28 weeks.

In certain embodiments, administering an effective amount of the hydroxypropyl β-cyclodextrin composition comprises administering to the human patient by intravenous infusion about 500 mg/kg to about 1000 mg/kg of the hydroxypropyl β-cyclodextrin composition, every 28 days, for a period of at least about 24 weeks. In certain embodiments, administering an effective amount of the hydroxypropyl β-cyclodextrin composition comprises administering to the human patient by intravenous infusion about 500 mg/kg to about 2000 mg/kg of the hydroxypropyl β-cyclodextrin composition, every 28 days, for a period of at least about 24 weeks.

In certain embodiments, the hydroxypropyl β-cyclodextrin composition is administered to the human patient by intravenous infusion over a period of at least about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, or about 8 hours. In certain embodiments, the hydroxypropyl β-cyclodextrin composition is administered to the human patient by intravenous infusion over a period of at least about 4 hours. In certain embodiments, the hydroxypropyl β-cyclodextrin composition is administered to the human patient by intravenous infusion over a period of at least about 6.5 hours.

In certain embodiments, the hydroxypropyl β-cyclodextrin composition is administered to the human patient by intravenous infusion over a period of about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, or about 8 hours. In certain embodiments, the hydroxypropyl β-cyclodextrin composition is administered to the human patient by intravenous infusion over a period of about 4 hours. In certain embodiments, the hydroxypropyl β-cyclodextrin composition is administered to the human patient by intravenous infusion over a period of about 6.5 hours.

In certain embodiments, the hydroxypropyl β-cyclodextrin composition comprises about 20% (w/v), about 21% (w/v), about 22% (w/v), about 23% (w/v), about 24% (w/v), about 25% (w/v), about 26% (w/v), about 27% (w/v), about 28% (w/v), about 29% (w/v), or about 30% (w/v) of a hydroxypropyl β-cyclodextrin. In certain embodiments, the hydroxypropyl β-cyclodextrin composition comprises about 25% (w/v) of a hydroxypropyl β-cyclodextrin.

In certain embodiments, the hydroxypropyl β-cyclodextrin is a hydroxypropyl β-cyclodextrin described herein. In certain embodiments, the hydroxypropyl β-cyclodextrin composition comprises a mixture of two or more hydroxypropyl β-cyclodextrin species. In certain embodiments, each of the two or more hydroxypropyl β-cyclodextrin species has a different degree of hydroxypropylation of the β-cyclodextrin ring. In certain embodiments, the mixture of the two or more hydroxypropyl β-cyclodextrin species has a molar substitution value from about 0.59 to about 1.14. In certain embodiments, the mixture of the two or more hydroxypropyl β-cyclodextrin species has a molar substitution value from about 0.59 to about 0.73. In certain embodiments, the mixture of the two or more hydroxypropyl β-cyclodextrin species has a molar substitution value from about 0.59 to about 0.8. In certain embodiments, the mixture of the two or more hydroxypropyl β-cyclodextrin species has a molar substitution value from about 0.8 to about 1.0. In certain embodiments, the hydroxypropyl β-cyclodextrin composition comprises about 0.2% w/w or less of unsubstituted β-cyclodextrin. In certain embodiments, the hydroxypropyl β-cyclodextrin composition comprises about 0.15% w/w or less of unsubstituted β-cyclodextrin. In certain embodiments, the hydroxypropyl β-cyclodextrin composition comprises about 0.16% w/w or less of unsubstituted β-cyclodextrin. In certain embodiments, the hydroxypropyl β-cyclodextrin composition comprises about 2.5% (w/w) or less of propylene glycol.

In certain embodiments, the method further comprises administering to the human patient a second therapeutic agent selected from the group consisting of donepezil, rivastigmine, galantamine, memantine, verubecestat, solanezumab, bapineuzumab, aducanumab, tideglusib, epothilone D and ABBV-8E12.

In certain embodiments, the method further comprises administering to the human patient a second therapeutic agent selected from the group comprising of a cholinesterase inhibitor, an NMDA receptor antagonist, a humanized antibody which targets tau protein, a humanized antibody which targets amyloid beta protein, and a BACE inhibitor.

In certain embodiments, the method further comprises administering to the human patient a second therapeutic agent, wherein the second therapeutic agent is selected from any therapeutic agent set forth in Table 1.

TABLE 1
Therapeutic agents for the treatment of Alzheimer's disease.

| | | | |
|---|---|---|---|
| ABBV-8E12 (anti-tau | AC-1204 (glucose stimulant) | ACI-24 (anti-Abeta | ACI-35 (anti-pTau vaccine) |
| antibody) | | vaccine) | |
| aducanumab (BIIB037) | AGB101 (levetiracetam low- | ALZ-801 (amyloid beta- | ALZT-OP1 (amyloid beta-protein |
| (amyloid beta mAb) | dose) | protein inhibitor) | inhibitor/inflammation mediator |
| | | | inhibitor) |
| AMG520/CNP520 (BACE1 | ANAVEX ™ 2-73 (M1 | AstroStem (mesenchymal | AUS-131 (nonhormonal estrogen |
| protein inhibitor) | muscarinic receptor agonist/ | stem cell therapy | receptor agonist) |
| | intracellular sigma 1 receptor | | |
| | agonist) | | |
| AVN-101 (serotonin 6 | AVN-322 (serotonin 6 receptor | AVP-786 | AXS-05 |
| receptor antagonist) | antagonist) | (dextromethorphan | (bupropion/dextromethorphan) |
| | | analogue/ultra- low dose | |
| | | quinidine) | |
| azeliragon (TTP488) | BAN2401 (anti-amyloid beta | Bexarotene (RXR- | BI 409306 (PDE9A inhibitor) |
| (RAGE antagonist) | mAb) | selective retinoid | |
| | | analogue) | |
| BIIB076 (anti-tau antibody) | BIIB092 (anti-extracellular tau | BNC375 (positive | BPN14770 (type 4 cyclic nucleotide |
| | antibody) | allosteric modulator) | phosphodiesterase inhibitor) |
| bryostatin 1 (protein kinase | CAD106 (amilomotide) (VLP | Corplex Donepezil | Corplex Memantine (memantine |
| C stimulant) | immunotherapy vaccine) | (donepezil transdermal | transdermal patch |
| | | patch | |
| CPC-201 | CPC-212 (next-generation | CPC-250 (next-generation | Crenezumab (anti-amyloid beta |
| (donepezil/solifenacin | acetylcholinesterase inhibitor) | acetylcholinesterase | antibody) |
| combination) | | inhibitor) | |
| CSP-1103 (amyloid beta- | CSTC1 (BAC) | CT1812 (amyloid beta | E2027 (PDE9 inhibitor) |
| protein inhibitor) | | oligomer receptor | |
| | | antagonist) | |
| E2609 (BACE1 protein | EVT302 (MAO-B inhibitor) | gantenerumab (amyloid | GC021109 (purinoceptor P2Y6 |
| inhibitor) | | beta-protein inhibitor) | agonist) |
| HSRx-888 (donepezil/food- | immune globulin/albumin | INP-102 intranasal | intepirdine (RVT-101) (serotonin 6 |
| based compound) | | | receptor antagonist) |
| IONIS-MAPTRx (tau- | JNJ-54861911 (BACE inhibitor) | JOT106 (oral capsule of | KPAX002-2 (proprietary version of |
| targeting protein) | | trans-resveratrol) | methylphenidate) |
| lanabecestat (BACE | LM11A-31 (p75 neutrophin | LMTX (tau protein | LY3002813 (N3pG-amyloid beta |
| inhibitor) | receptor) | aggregation inhibitor/ | antibody) |
| | | TDP-43 aggregation | |
| | | inhibitor) | |
| LY3202626 (BACE | LY3303560 (tau antibody) | MI agonist (selective M1 | MEDI1814 (anti-amyloid beta 42 |
| inhibitor) | | receptor agonist) | mAb) |
| mesenchymal stem cell | MP-101 (mGluR2/mGluR3 | MSDC-0160 (mTOT | NBXT-001 + Nobilis ™ inhalation |
| therapy | agonist) | modulator) | device (NMDA receptor antagonist) |
| neflamapimod (VX-745) | NGP 555 (gamma secretase | nilvadipine soluble | NPT088 (GAIM Ig fusion targeting |
| (p38 mitogen-activated | complex modulator) | amyloid reducing/clearing | amyloid-β, tau, a-synuclein) |
| protein kinase inhibitor) | | agent) | |
| Nuplazid ® pimavanserin | PF-05251749 (casein kinase 1 | PF-06648671 (gamma | PF-06751979 (enzyme inhibitor) |
| | delta/epsilon) | secretase complex | |
| | | modulator) | |
| pioglitazone (low-dose) | piromelatine (melatonin agonist) | Posiphen ® R-phenserine | Rexulti ® brexpiprazole |
| (PPARγ agonist) | | | |
| RG6100 (tau protein | RVT-103 + RVT-104 | SAR228810 (anti- | selective BACE 1 inhibitor |
| inhibitor) | (QAAM + cholinesterase | protofibrillar AB mAb) | |
| | inhibitor) | | |
| solanezumab (amyloid beta | SUVN-502 (serotonin 6 receptor | SUVN-D4010 (serotonin 4 | SUVN-G3031 (histamine H3 |
| protein inhibitor) | antagonist) | receptor agonist) | receptor antagonist) |
| T-817MA (amyloid beta- | T3D-959 (PPAR-delta/gamma | TAK-071 (muscarinic M1 | TPI 287 (next-generation taxane) |
| protein inhibitor) | agonist) | receptor modulator) | |
| UB-311 (anti-amyloid | UE-2343 (11β-HSD1 inhibitor) | verubecestat (MK-8931) | |
| endobody vaccine) | | (BACE1 protein inhibitor) | |

### Pharmaceutical Compositions

In one aspect, the disclosure provides pharmaceutical compositions for the treatment of early Alzheimer's disease in a human patient in need thereof, the pharmaceutical composition generally comprising an effective amount of a hydroxypropyl β-cyclodextrin composition disclosed herein and one or more pharmaceutically acceptable excipients.

In some embodiments, a pharmaceutical composition described herein can comprise about 20,000 mg, about 21,000 mg, about 22,000 mg, about 23,000 mg, about 24,000 mg, about 25,000 mg, about 26,000 mg, about 27,000 mg, about 28,000 mg, about 29,000 mg, or about 30,000 mg, of the hydroxypropyl β-cyclodextrin composition.

In some embodiments, the concentration of the hydroxypropyl β-cyclodextrin in a pharmaceutical composition described herein is about 100 mg/mL, about 150 mg/mL, about 200 mg, mL, about 250 mg/mL, about 300 mg/mL, or about 350 mg/mL.

In certain embodiments, the pharmaceutical composition comprises a solution comprising about 5% (w/v) to about 40% (w/v), about 10% (w/v) to about 40% (w/v), about 15% (w/v) to about 40% (w/v), about 20% (w/v) to about 40% (w/v), about 25% (w/v) to about 40% (w/v), about 30% (w/v) to about 40% (w/v), about 35% (w/v) to about 40% (w/v), about 5% (w/v) to about 35% (w/v), about 5% (w/v) to about 30% (w/v), about 5% (w/v) to about 25% (w/v), about 5% (w/v) to about 20% (w/v), about 5% (w/v) to about 15% (w/v), about 5% (w/v) to about 10% (w/v), about 10% (w/v) to about 35% (w/v), about 10% (w/v) to about 30% (w/v), about 10% (w/v) to about 25% (w/v), about 10% (w/v) to about 20% (w/v), about 10% (w/v) to about 15% (w/v), about 15% (w/v) to about 35% (w/v), about 15% (w/v) to about 30% (w/v), about 15% (w/v) to about 25% (w/v), about 15% (w/v) to about 20% (w/v), about 20% (w/v) to about 35% (w/v), about 20% (w/v) to about 30% (w/v), about 20% (w/v) to about 25% (w/v), about 25% (w/v) to about 35% (w/v), about 25% (w/v) to about 30% (w/v), or about 30% (w/v) to about 35% (w/v) of one or more hydroxypropyl β-cyclodextrin species.

In certain embodiments, the hydroxypropyl β-cyclodextrin composition comprises a 5% (w/v), 10% (w/v), 15% (w/v), 20% (w/v), 25% (w/v), 30% (w/v), 35% (w/v) or 40% (w/v) aqueous solution of one or more hydroxypropyl β-cyclodextrin species. In some embodiments, the effective amount of the hydroxypropyl β-cyclodextrin composition comprises a 25% (w/v) solution of one or more hydroxypropyl β-cyclodextrin species. In some embodiments, the 25% (w/v) solution of one or more hydroxypropyl β-cyclodextrin species is a 25% (w/v) aqueous solution of one or more hydroxypropyl β-cyclodextrin species.

In certain embodiments, the pharmaceutical composition comprises a 5% (w/v), 10% (w/v), 15% (w/v), 20% (w/v), 25% (w/v), 30% (w/v), 35% (w/v) or 40% (w/v) aqueous solution of one or more hydroxypropyl β-cyclodextrin species. In some embodiments, the effective amount of the pharmaceutical composition comprises a 25% (w/v) solution of one or more hydroxypropyl β-cyclodextrin species. In some embodiments, the 25% (w/v) solution of one or more hydroxypropyl β-cyclodextrin species is a 25% (w/v) aqueous solution of one or more hydroxypropyl β-cyclodextrin species.

In some embodiments, the one or more pharmaceutically acceptable excipients is selected from the group comprising a diluent, a buffering agent, a preservative, a stabilizer, a solubilizing agent or any combination thereof.

In certain embodiments, the pharmaceutical composition may be formulated for administration as a liquid dosage form suitable for intracavitary, intradermal, intramuscular, intrathecal, intravenous, subcutaneous, or intracerebroventricular administration.

In certain embodiments, a liquid dosage form of a pharmaceutical composition as described herein further comprises a diluent. In some embodiments, the insert diluent is a saline solution.

In certain embodiments, a liquid dosage form of a pharmaceutical composition as described herein further comprises a buffering agent. For example, suitable buffering agents for use with the present disclosure include, but are not limited to, both organic and inorganic acids and salts thereof, such as citrate buffers (e.g., monosodium citrate-disodium citrate mixture, citric acid-trisodium citrate mixture, citric acid-monosodium citrate mixture), succinate buffers (e.g., succinic acid-monosodium succinate mixture, succinic acid-sodium hydroxide mixture, succinic acid-disodium succinate mixture), tartrate buffers (e.g., tartaric acid-sodium tartrate mixture, tartaric acid-potassium tartrate mixture, tartaric acid-sodium hydroxide mixture), fumarate buffers (e.g., fumaric acid-monosodium fumarate mixture, fumaric acid-disodium fumarate mixture, monosodium fumarate-disodium fumarate mixture), gluconate buffers (e.g., gluconic acid-sodium gluconate mixture, gluconic acid-sodium hydroxide mixture, gluconic acid-potassium gluconate mixture), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris may be used.

In certain embodiments, a liquid dosage form of a pharmaceutical composition as described herein further comprises a pH adjuster. For example, suitable pH adjusters include, but are not limited to, 1N sodium hydroxide and 37% hydrochloric acid (HCl).

In certain embodiments, a liquid dosage form of a pharmaceutical composition as described herein further comprises a preservative. For example, suitable preservatives for use with the present disclosure include, but are not limited to phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens (e.g., methyl or propyl paraben, catechol, resorcinol, cyclohexanol, and 3-pentanol).

In certain embodiments, a liquid dosage form of a pharmaceutical composition as described herein further comprises a stabilizer. For example, suitable stabilizers include, but are not limited to polyhydric sugar alcohols, trihydric or higher sugar alcohols, amino acids, organic sugars or sugar alcohols, polyvinylpyrrolidone monosaccharides, trisaccacharides, polysaccharides, proteins, sulfur containing reducing agents, amino acid polymers and polyethylene glycol.

In certain embodiments, a liquid dosage form of a pharmaceutical composition as described herein further comprises a solubilizing agent. In some embodiments, the solubilizing agent is an ionic surfactant. Examples of non-ionic surfactants include, but are not limited to, polysorbates, polyoxamers, pluronic polyols, and polyoxyethylene sorbitan monoethers.

In certain embodiments, pharmaceutical compositions of the hydroxypropyl β-cyclodextrin compositions disclosed herein can be prepared for storage as lyophilized formulations or aqueous solutions by mixing the hydroxypropyl β-cyclodextrin composition with optional pharmaceutically-acceptable carriers, excipients or stabilizers typically employed in the art (e.g., buffering agents, stabilizing agents, preservatives, isotonifiers, non-ionic detergents, antioxidants, and other miscellaneous additives).

In certain embodiments, a pharmaceutical composition comprising a hydroxypropyl α-cyclodextrin composition disclosed herein is chemically and/or physically stable at RT (15-25° C.) for about 6 months, about 1 year, about 1 year and 6 months, about 2 years, about 2 years and 6 months, about 3 years, about 3 years and 6 months, about 4 years, about 4 years and 6 months, or about 5 years.

In certain embodiments, the pharmaceutical compositions described herein further comprise a second therapeutic agent. In some embodiments, the second therapeutic agent is indicated to treat early Alzheimer's disease. In some embodiments, the second therapeutic agent is selected from the group consisting of donepezil, rivastigmine, galantamine, memantine, verubecestat, solanezumab, bapineuzumab, aducanumab, tideglusib, epothilone D and ABBV-8E12. In some embodiments, the second therapeutic agent is selected from the group consisting of a cholinesterase inhibitor, an NMDA receptor antagonist, a humanized antibody which targets tau protein, a humanized antibody which targets amyloid beta protein, and a BACE inhibitor. In some embodiments, the second therapeutic agent is selected from the group consisting of Aricept®, Namenda®, donepezil, memantine, Excelon®, Namenda XR®, galantamine, Aricept® ODT, rivastigmine, vitamin e, Razadyne® ER, donepezil/memantine, Razadyne®, Namzaric®, Alpha E®, Hydergine®, ergoloid mesylates, Aqua-E®, Aqua Gem-E®, etanercept, Reminyl®, Vita-Plus E natural, Aquasol E®, Aquavite-E® and E-400 clear.

In some embodiments, the second therapeutic agent is selected from any therapeutic agent set forth in Table 1.

In certain embodiments, the second therapeutic agent is selected from the group consisting of ABBV-8E12 (anti-tau antibody), AC-1204 (glucose stimulant), ACI-24 (anti-Abeta vaccine), ACI-35 (anti-pTau vaccine), aducanumab (BIIB037) (amyloid beta mAb), AGB101 (levetiracetam low-dose), ALZ-801 (amyloid beta-protein inhibitor), ALZT-OP1 (amyloid beta-protein inhibitor/inflammation mediator inhibitor), AMG520/CNP520 (BACE1 protein inhibitor), ANAVEX™ 2-73 (MI muscarinic receptor agonist/intracellular sigma 1 receptor agonist), AstroStem (mesenchymal stem cell therapy, AUS-131 (nonhormonal estrogen receptor agonist), AVN-101 (serotonin 6 receptor antagonist), AVN-322 (serotonin 6 receptor antagonist), AVP-786 (dextromethorphan analogue/ultra-low dose quinidine), AXS-05 (bupropion/dextromethorphan), azeliragon (TTP488) (RAGE antagonist), BAN2401 (anti-amyloid beta mAb), Bexarotene (RXR-selective retinoid analogue), BI 409306 (PDE9A inhibitor), BIIB076 (anti-tau antibody), BIIB092 (anti-extracellular tau antibody), BNC375 (positive allosteric modulator), BPN14770 (type 4 cyclic nucleotide phosphodiesterase inhibitor), bryostatin 1 (protein kinase C stimulant), CAD106 (amilomotide) (VLP immunotherapy vaccine), Corplex Donepezil (donepezil transdermal patch, Corplex Memantine (memantine transdermal patch, CPC-201 (donepezil/solifenacin combination), CPC-212 (next-generation acetylcholinesterase inhibitor), CPC-250 (next-generation acetylcholinesterase inhibitor), Crenezumab (anti-amyloid beta antibody), CSP-1103 (amyloid beta-protein inhibitor), CSTCI (BAC), CT1812 (amyloid beta oligomer receptor antagonist), E2027 (PDE9 inhibitor), E2609 (BACE1 protein inhibitor), EVT302 (MAO-B inhibitor), gantenerumab (amyloid beta-protein inhibitor), GC021109 (purinoceptor P2Y6 agonist), HSRx-888 (donepezil/food-based compound), immune globulin/albumin, INP-102 intranasal, intepirdine (RVT-101) (serotonin 6 receptor antagonist), IONIS-MAPTRx (tau-targeting protein), JNJ-54861911 (BACE inhibitor), JOT106 (oral capsule of trans-resveratrol), KPAX002-2 (proprietary version of methylphenidate), lanabecestat (BACE inhibitor), LM11A-31 (p75 neutrophin receptor), LMTX (tau protein aggregation inhibitor/TDP-43 aggregation inhibitor), LY3002813 (N3pG-amyloid beta antibody), LY3202626 (BACE inhibitor), LY3303560 (tau antibody), MI agonist (selective MI receptor agonist), MEDI1814 (anti-amyloid beta 42 mAb), mesenchymal stem cell therapy, MP-101 (mGluR2/mGluR3 agonist), MSDC-0160 (mTOT modulator), NBXT-001+Nobilis™ inhalation device (NMDA receptor antagonist), neflamapimod (VX-745) (p38 mitogen-activated protein kinase inhibitor), NGP 555 (gamma secretase complex modulator), nilvadipine soluble amyloid reducing/clearing agent), NPT088 (GAIM Ig fusion targeting amyloid-β, tau, a-synuclein), Nuplazid® pimavanserin, PF-05251749 (casein kinase 1 delta/epsilon), PF-06648671 (gamma secretase complex modulator), PF-06751979 (enzyme inhibitor), pioglitazone (low-dose) (PPARγ agonist), piromelatine (melatonin agonist), Posiphen® R-phenserine, Rexulti® brexpiprazole, RG6100 (tau protein inhibitor), RVT-103+RVT-104 (QAAM+cholinesterase inhibitor), SAR228810 (anti-protofibrillar AB mAb), selective BACE 1 inhibitor, solanezumab (amyloid beta protein inhibitor), SUVN-502 (serotonin 6 receptor antagonist), SUVN-D4010 (serotonin 4 receptor agonist), SUVN-G3031 (histamine H3 receptor antagonist), T-817MA (amyloid beta-protein inhibitor), T3D-959 (PPAR-delta/gamma agonist), TAK-071 (muscarinic MI receptor modulator), TPI 287 (next-generation taxane), UB-311 (anti-amyloid endobody vaccine), UE-2343 (11β-HSD1 inhibitor), verubecestat (MK-8931) (BACE1 protein inhibitor), or combinations thereof.

In certain embodiments, the pharmaceutical compositions of the present disclosure are administered to the subject by intracavitary, intradermal, intramuscular, intrathecal, intravenous, subcutaneous, or intracerebroventricular administration.

### Kits

In various embodiments, the invention provides kits for treating early Alzheimer's disease in a human patient in need thereof. In some embodiments, the kit generally comprises: i) instructions for administering the hydroxypropyl β-cyclodextrin compositions or pharmaceutical compositions described herein to a human patient suffering from early Alzheimer's disease, and ii) a hydroxypropyl β-cyclodextrin composition or a pharmaceutical composition as described herein. In some embodiments, the kit may comprise one or more unit dosage forms containing an amount of a hydroxypropyl β-cyclodextrin composition, or a pharmaceutical composition as described herein that is effective for treating early Alzheimer's disease in the human patient.

In certain embodiments, the kit comprises: i) instructions for administering the hydroxypropyl β-cyclodextrin compositions or pharmaceutical compositions described herein to a human patient in need thereof, and (ii) one or more 100 mL vials comprising 25% (w/v) of a hydroxypropyl β-cyclodextrin composition as described herein. In some embodiments, the hydroxypropyl β-cyclodextrin composition is the Trappsol® Cyclo™ hydroxypropyl β-cyclodextrin composition.

In some embodiments, the kit further comprises one or more selected from the group comprising a sterile syringe, a sterile needle, a sterile IV bag, an infusion pump or any combination thereof.

The description above describes multiple aspects and embodiments of the present invention, including hydroxypropyl β-cyclodextrin compositions, methods of using a hydroxypropyl β-cyclodextrin compositions to treat a subject with early Alzheimer's disease, pharmaceutical compositions comprising a hydroxypropyl β-cyclodextrin compositions, and kits. The patent application specifically contemplates all combinations and permutations of the aspects and embodiments.

### EXAMPLES

In order that the disclosure described herein may be more fully understood, the following examples are set forth. The examples described in this application are offered to illustrate the compositions and methods provided herein and are not to be construed in any way as limiting their scope.

### Abbreviations

A amyloid burden
Aβ amyloid-beta
Aβ40 amyloid-beta 40
Aβ42 amyloid-beta 42
AD Alzheimer's disease
ADAS-Cog-14 Alzheimer's disease assessment scale-cognitive subscale 14
ADCS-ADL Alzheimer's disease cooperative study-activities of daily living
ADCS-CGIC Alzheimer's disease cooperative study-clinical global impression of change
AE adverse event
APP amyloid precursor protein processing
ApoE apolipoprotein E
APS amyloid probability score
APOE apolipoprotein E
aPTT activated partial thromboplastin
ARIA amyloid-related imaging abnormality
ARIA-E amyloid-related imaging abnormality-edematous
ARIA-H amyloid-related imaging abnormality-hemorrhagic
ATN amyloid/tau/neurodegeneration
BACE β-site APP cleaving enzyme
CDR clinical dementia rating scale
CDR-SB clinical dementia rating scale sum of boxes
CNS central nervous system
CSF cerebrospinal fluid
C-SSRS Columbia-suicide severity rating scale
CT computerized tomography
DS/DSₐ average degree of substitution
DSMB data and safety monitoring board
DSM-5 diagnostic and statistical manual of mental disorders, 5th Edition
EAD early Alzheimer's disease
ECG electrocardiogram
eGFR estimated glomerular filtration rate
EOS end of study
EOT end of treatment
EOS end of study
ET early termination
FDA Food and Drug Administration
GDS geriatric depression scale-short form
GDS-SF geriatric depression scale-short form
HDL high-density lipoprotein
HIS modified Hachinski ischemia score
HPβCD hydroxypropyl-beta-cyclodextrin
INR international normalized ratio
IV intravenous
LE late endosomes
LDL low-density lipoprotein
LY late lysosomes
MCI mild cognitive impairment
MDD major depressive disorder
MDRD modification of diet in renal disease
MMSE-2:SV mini-mental state examination-2 standard version
MRI magnetic resonance imaging
MS/MSₐ average molar substitution
NfL neurofilament light chain
NIA-AA National Institute on Aging-Alzheimer's Association
NMDA N-methyl-D-aspartate
PD pharmacodynamic
PET positron emission tomography
PK pharmacokinetics
PT prothrombin time
SoA schedule of assessments
TdP Torsades de Pointes
TEAE treatment-emergent adverse event
TSH thyroid-stimulating hormone
USP United States Pharmacopeia
WFI water for injection
WOCBP women of childbearing potential

### Example 1. A Randomized, Placebo-Controlled, Double-Blind, Parallel-Group, 6-Month Study to Evaluate the Safety, Tolerability, and Potential Efficacy of Monthly Trappsol® Cyclo™ (Hydroxypropyl β-Cyclodextrin, HPβCD) Infusions in Patients With Early Alzheimer's Disease

### 1. Objectives

Assess the safety and tolerability of Trappsol® Cyclo™ over 24 weeks in patients with early Alzheimer's disease (EAD).

Explore the potential efficacy of Trappsol® Cyclo™ over 24 weeks in patients with EAD.

Determine the pharmacokinetics (PK) of Trappsol® Cyclo™ over 24 weeks in patients with EAD.

### 2. Study Design

This is a randomized, placebo-controlled, double-blind, parallel-group study that assesses the safety, tolerability, and potential efficacy of Trappsol® Cyclo™ in patients with EAD as defined according to the FDA Guidance for Industry on Early Alzheimer's Disease: Developing Drugs for Treatment. [Vellas B, Bateman R, Blennow K, et al. Endpoints for pre-dementia AD trials: a report from the EU/US/CTAD Task Force. J Prev Alzheimers Dis. 2015; 2(2):128-135.]

The study enrolls approximately 120 (30 patients/treatment arm) male and female patients aged 50 to 80 years at screening with characteristic pathophysiologic changes of Alzheimer's disease (AD) who meet National Institute on Aging-Alzheimer's Association (NIA-AA) or FDA Guidance for Industry on Early Alzheimer's Disease: Developing Drugs for Treatment criteria for either AD with mild cognitive impairment (MCI) or mild AD collectively known as EAD (Stages 3 and 4). [Cummings J. The National Institute on Aging-Alzheimer's Association Framework on Alzheimer's disease: application to clinical trials. Alzheimers Dement. 2019; 15(1):172-178., Kantner I, Erben R G. Long-term parenteral administration of 2-hydroxypropyl β-cyclodextrin causes bone loss. Toxicol Pathol. 2012; 40(5):742-50., Vellas B, Bateman R, Blennow K, et al. Endpoints for pre-dementia AD trials: a report from the EU/US/CTAD Task Force. J Prev Alzheimers Dis. 2015; 2(2):128-135.] Enrolled patients have evidence of progressive cognitive decline in the last year as determined by serial cognitive test scores, if available, or patient or informant/caregiver/study partner (hereafter called caregiver) report as documented by the Investigator.

Due to the nature of EAD, patients identify a primary caregiver prior to enrollment in the study who assists the patient with study participation. The patient is accompanied to each clinic visit by their caregiver. The caregiver has face-to-face contact with the patient for a minimum of approximately 10 hours per week spread over 2 to 5 days during the week (e.g., 2 hours/day 5 days a week or 5 hours/day twice a week). The legally authorized representative may be the caregiver. The caregiver need not be a family member.

During the screening period (up to 42 days prior to Visit [V] 2/Baseline), all potential patients and their primary caregiver provide written informed consent to participate in the study prior to any study-related assessments being performed. A blood sample is collected from all patients at the screening visit (V1) for a Precivity AD™ blood test and/or clinical laboratory assessments.

Patients also have magnetic resonance imaging (MRI) within the 12 months prior to the screening visit (V1) to confirm the diagnosis of EAD without any other clinically significant pathologies. A computerized tomography (CT) scan may only be substituted if the patient has an absolute contraindication for MRI.

Although the FDA Guidance for Industry on Early Alzheimer's Disease: Developing Drugs for Treatment [Vellas B, Bateman R, Blennow K, et al. Endpoints for pre-dementia AD trials: a report from the EU/US/CTAD Task Force. J Prev Alzheimers Dis. 2015; 2(2):128-135.] remains silent on the specific biomarkers used to satisfy recent consensus statements regarding amyloid/tau/neurodegeneration (ATN) criteria, the protocol provides additional requirements regarding detection of amyloid burden (A).

Specifically, to support the diagnostic criteria based upon relevant guidance of the NIA-AA criteria, [Cummings J. The National Institute on Aging-Alzheimer's Association Framework on Alzheimer's disease: application to clinical trials. Alzheimers Dement. 2019; 15 (1):172-178., Kantner I, Erben RG. Long-term parenteral administration of 2-hydroxypropyl β-cyclodextrin causes bone loss. Toxicol Pathol. 2012; 40(5):742-50.] all eligible patients should need to undergo lumbar puncture and have a CSF biomarker profile in accordance with the diagnosis of AD or a PrecivityAD blood test with high amyloid probability score (APS [58-100]), during the Screening Period.

Patients that meet enrollment criteria based on results from the Precivity AD blood test and other eligibility criteria, also exhibit evidence of cerebral amyloid-beta (Aβ) pathology prior to enrollment at the baseline visit (V2) based on cerebrospinal fluid (CSF) analysis evaluating the levels Aβ42/40; or patients with MCI need to meet the criteria of CSF Aβ42/40 ratio<0.72, and patients with EAD need to meet the criteria of Aβ42/40 ratio<0.72 and a CSF P-tau>50 pg/mL.

A Clinical Dementia Rating Scale Sum of Boxes (CDR-SB), Mini-Mental State Examination-2: Standard Version (MMSE-2:SV), Alzheimer's Disease Cooperative Study-Clinical Global Impression of Change (ADCS-CGIC), Alzheimer's Disease Cooperative Study-Activities of Daily Living (ADCS-ADL), and Columbia-Suicide Severity Rating Scale (C-SSRS) are obtained to support the AD diagnosis based on diagnostic guidelines of the NIA-AA criteria [Cummings J. The National Institute on Aging-Alzheimer's Association Framework on Alzheimer's disease: application to clinical trials. Alzheimers Dement. 2019; 15(1):172-178., Kantner I, Erben RG. Long-term parenteral administration of 2-hydroxypropyl β-cyclodextrin causes bone loss. Toxicol Pathol. 2012; 40(5):742-50.] as well as a physical examination, vital signs, and 12-lead electrocardiogram (ECG). The Geriatric Depression Scale-Short Form (GDS-SF) and Modified Hachinski Ischemia Scale (HIS) are used to exclude underlying diagnoses. Prior and current medications are recorded. Additional required assessments are itemized in the Schedule of Assessments (SoA, Table 2).

TABLE 2
Schedule of Assessments

| Visit | | | | | | | | |
|---|---|---|---|---|---|---|---|---|
| 1 | 2 | | | | | | 8/EOT | 9 |
| Screening | Baseline | 3 | 4 | 5 | 6 | 7 | EOS | Follow up |
| Weeks | | | | | | | | |
| −7 through | | | | | | | | 26 |
| 0 (42 days) | 0 | 4 | 8 | 12 | 16 | 20 | 24 | (±14 days) |

General Assessments

| | | |
|---|---|---|
| Informed consent | X | |
| Demographics | X | |
| Medical history | X | |
| Inclusion/exclusion criteria | X | X |

Eligibility Assessments

| | | |
|---|---|---|
| Precivity AD ™ blood test | X | X |
| HIS | X | |
| GDS-SF | X | |
| CSF levels of Aβ42/40¹ | X | X |

Efficacy Assessments (Cognitive assessments should be performed
at the same time of day (±1 hour) throughout the trial.)

| | | | | |
|---|---|---|---|---|
| ADAS-Cog-14 | | X | X | X |
| CDR-SB | X | X | | X |
| MMSE-2:SV | X | X | X | X |
| ADCS-CGIC | X | X | X | X |
| ADCS-ADL | X | X | X | X |

Safety Assessments

| | | | | | | | | | |
|---|---|---|---|---|---|---|---|---|---|
| Physical examination | X | | | | | | | X | |
| Vital signs² | X | X | X | X | X | X | X | X | X |
| Triplicate 12-lead ECG³ | X | X | X | X | X | | | X | |
| C-SSRS | X | X | X | X | X | X | X | X | |
| Safety clinical laboratory | X | X | | | X | | | X | |
| tests⁴ | | | | | | | | | |
| PK sampling⁵ | | X | X | X | X | | | X | |
| Urinalysis | X | X | | | X | | | X | |
| POC urine pregnancy test⁶ | X | X | X | X | X | X | X | X | |
| Urine drug screen⁷ | X | X | | | | | | | |
| PD-blood⁸/plasma/ApoE | | X | X | X | X | X | X | X | |
| measures | | | | | | | | | |
| MRI (ARIA)⁹ | X | | | X | | X | | X | |
| Audiological examination¹⁰ | X | | | | X | | | X | |
| Prior/concomitant | X | X | X | X | X | X | X | X | X |
| medications | | | | | | | | | |
| AE | | X | X | X | X | X | X | X | X |

Study Drug Assessments

| | | | | | | | |
|---|---|---|---|---|---|---|---|
| Randomization | X | | | | | | |
| Study drug infusion¹¹ | X | X | X | X | X | X | X |

Note:
On a case-by-case basis with permission of the Sponsor in consultation with the Medical Monitor and Investigator, if the patient cannot complete all assessments in 1 study visit day, may extend the study visit over 2 days. The efficacy assessments and certain safety assessments (vital signs, AEs) are to be completed on the first day, with the IMP IV infusion, safety assessments (labs, vital signs, ECG, AEs, safety scales), and PK sampling completed on the second day.
¹After the patient meets enrollment criteria based on results from the PrecivityAD blood test and other eligibility criteria, a CSF analysis evaluating the levels of Aβ42/40 must be performed to confirm the diagnosis of Alzheimer's disease. If the CSF assessments was already performed prior to Screening, repeating these tests is not necessary provided that results from the PrecivityAD blood test met enrollment criteria. All patients will need to be willing to also consent for a lumbar puncture and CSF analysis at the EOT/EOS visit. Prior to lumbar puncture, blood coagulation tests (prothrombin time [PT]/international normalized ratio [INR], and activated partial thromboplastin time [aPTT]) will be performed. Platelet measurements are included among clinical laboratory safety tests for the Screening Visit. Per Investigator's discretion, additional coagulation tests prior to lumbar puncture could be performed for a patient who has an abnormal coagulation result or for reason of increased bleeding risk.
²Vital signs will include body temperature, respiratory rate, body weight, supine and sitting radial pulse rates, and sitting systolic and diastolic blood pressures. Sitting recordings are to be made after the patient has been sitting for at least 3 minutes with their feet squarely on the floor and arm relaxed, bent at the elbow. The method of obtaining body temperature will be per sites' standard practice but should be obtained by the same method for a given patient throughout the study. Vital signs will be measured prior to start of infusion. Height for calculation of body mass index will be captured during physical examinations.
³Standard, triplicate 12-lead ECGs will be evaluated at each center for the presence of clinically relevant abnormalities. The ECGs will be obtained after the patient has been quiet and supine for approximately 3 minutes using the same ECG machine each time. The ECGs will be evaluated by a medically qualified investigator at each center for the presence of clinically relevant abnormalities during infusion. A triplicate ECG will be obtained at Screening to confirm QTcF <450 ms for males and <460 ms for females; for all other visits as indicated, ECG readings will be completed prior to start of infusion, at +60 minutes following onset of infusion, and immediately following completion of infusion. At baseline, triplicate ECGs will be collected at 3 different timepoints (−60, −45 and −30 minutes) starting −60 minutes prior to the first dose administration (Week 0). Triplicate 12-lead ECGs will be time-matched with PK sampling (EGC tracing should be followed by PK sampling, ideally within 2 minutes) at Week 0 (Baseline), Week 4, and Week 8.
⁴Safety laboratory tests include hematology (red blood cell count, white blood cell count, lymphocytes, neutrophils, eosinophils, basophils, monocytes, hemoglobin, hematocrit, and platelets), chemistry (alanine aminotransferase, aspartate aminotransferase, total bilirubin, direct bilirubin, γ-glutamyl transferase, blood urea nitrogen, total cholesterol, LDL cholesterol, HDL cholesterol, triglycerides, creatinine, eGFR by MDRD, alkaline phosphatase, lactate dehydrogenase, sodium, potassium, calcium, chloride, albumin, uric acid, and glucose), C-terminal telopeptide, and urinalysis (macroscopic analysis with microscopic analysis only when indicated by dipstick). Analysis will include color, turbidity, specific gravity, pH, glucose, protein, ketones, urobilinogen, bilirubin, blood nitrite, and leukocyte esterase. At Screening, laboratory tests will also include serology testing (HIV-1, HIV-2, hepatitis C surface antigen, and hepatitis C virus antibodies), coagulation (PT/INR and aPTT), TSH, T3 and T4 in patients with a clinical suspicion of severe hypothyroidism, eGFR by MDRD, and Vitamin B12. Laboratory tests will be completed prior to start of infusion.
⁵PK sampling will be completed in all patients prior to the start of the study drug infusion, 1 and 3 hours into the infusion, upon completion of infusion, 50 to 60 minutes post-infusion, and 2 hours post-infusion at Baseline (Week 0, V2), Week 4 (V3) and Week 8 (V4). PK sampling for trough levels will be obtained prior to the start of study drug infusion at Week 12 (V5), and Week 24 (V8). Participation in PK procedures for ET patients is not required unless a safety observation prompted discontinuation of study medication. PK sampling will be time-matched with triplicate 12-lead ECGs (tracing should be followed by PK sampling, ideally within 2 minutes) at Week 0 (Baseline), Week 4, and Week 8.
⁶A POC urine pregnancy test will be performed for female patients of child-bearing potential at each visit. Any positive POC urine pregnancy test will be confirmed with a serum pregnancy test prior to the patient being continued in the study.
⁷A urine drug screen will be performed at Screening (V1) and prior to infusion at Baseline (V2).
⁸Any residual blood and/or plasma from PD marker analysis will be stored for subsequent analysis.
⁹MRI assessments are planned for both for ARIA-E and ARIA-H; results will be evaluated by the DSMB.
¹⁰Patients will complete auditory assessments (e.g., pure tone average/pure tone 1 to 8 kHz audiometry or auditory-evoked potential/auditory brainstem response) during Screening (V1), within a week prior to each study drug administration as of Visit 3 (with audiometry results available for the investigator's review before the next study drug administration), and at Week 24 (EOT/EOS, V8). Furthermore, additional assessments may be scheduled at Investigator's discretion based on a patient's report of hearing loss. Patients will be prompted prior to study drug infusion and at the Follow-up Phone Call to report any hearing loss.
¹¹A dose of study drug will be administered in the clinic after all assessments are performed every 4 weeks by IV infusion over 4 hours through Week 24. If agreed between the Investigator, Sponsor, and the patient/caregiver, the study drug may be administered at home by appropriately trained personnel. The minimum interval between infusions is 25 days, and the maximum interval, 42 days; exceptions to this guidance should be reviewed by the Medical Monitor.

At the baseline visit (V2), patients meeting all eligibility criteria are randomly assigned to receive 1 of 3 study treatments in a 1:1:1:1 ratio:
I. 500 mg/kg Trappsol® Cyclo™
II. 1000 mg/kg Trappsol® Cyclo™
III. 2000 mg/kg Trappsol® Cyclo™
IV. Placebo

A dose of study drug is administered every 4 weeks by intravenous infusion over 4 hours through Week 24. Each patient receives up to 7 doses of study drug.

The patient and their caregiver visit the clinic every 4 weeks (V2 through V8). The caregiver does not need to be present for the entire visit. Safety is evaluated throughout the study by repeated physical examinations, vital signs, clinical laboratory tests, 12-lead ECG, C-SSRS, and recording of concomitant medications and adverse events (AEs). Potential efficacy of the study drug is assessed throughout the study by collection of the Alzheimer's Disease Assessment Scale-Cognitive Subscale 14 (ADAS-Cog-14), CDR-SB and MMSE-2: SV., Specific assessments at each visit are itemized in the SoA (Table 2).

At a follow-up visit (V9) 2 weeks after the last dose of study drug, safety assessments are obtained. Specific assessments at each visit are itemized in the SoA (Table 2).

MRI assessments are planned over the 6 months of treatment both for amyloid-related imaging abnormality-edematous (ARIA-E) and amyloid-related imaging abnormality-hemorrhagic (ARIA-H). Scanning is scheduled at screening (V1), and 8 weeks (V4), 16 weeks (V6), and 6 months (V8) after the start of double-blind study medication for each patient. Local reading for these radiological assessments always occurs as part of safety evaluations; central reading also may be effected. A Data and Safety Monitoring Board (DSMB) oversees results of these assessments in fully blinded fashion to determine whether amyloid-related imaging abnormality (ARIA) are detected in patients randomized to active therapy.

The safety of study participants is evaluated throughout the study by repeated physical examinations, vital signs, safety laboratory tests, 12-lead ECGs, the emergence of ARIA, and assessments of treatment-emergent adverse events (TEAEs). Periodic safety reviews are conducted during the study. The frequency of these assessments based upon the percentage of patients and time in study is defined in a DSMB charter developed prior to study initiation.

PK sampling is completed in all patients prior to the start of the study drug infusion, 1 and 3 hours into the infusion, upon completion of infusion, 50 to 60 minutes post-infusion, and 2 hours post-infusion at baseline (Week 0, V2), Week 4 (V3), Week 8 (V4). Sampling for potential analysis of PK trough levels is completed in all patients prior to the start of the study drug infusion at Week 12 (V5), and Week 24 (V8). ECG triplicate tracings are time-matched with PK samples at Week 0 (Baseline), Week 4, and Week 8.

### 3. Duration of Treatment

The study consists of 3 periods: screening period up to 42 days, treatment period up to 24 weeks, and a safety follow-up period of 2 weeks. The total duration of a patient's participation in the study is up to 32 weeks.

### 4. Planned Sample Size and Treatment Groups

The planned sample size is approximately 120 randomized patients. The study includes 3 arms: 500 mg/kg Trappsol® Cyclo™, 1000 mg/kg or 2000 mg/kg Trappsol® Cyclo™, or placebo at a 1:1:1:1 ratio (30 patients/arm). All patients receive the study drug or placebo intravenously.

### 5. Target Population

Patients with EAD.

### 6. Study Population

The study enrolls approximately 120 generally healthy male and female patients≥50 and ≤80 years of age, with EAD (patients with MCI due to AD and mild AD) according to the NIA-AA criteria, [Cummings J. The National Institute on Aging-Alzheimer's Association Framework on Alzheimer's disease: application to clinical trials. Alzheimers Dement. 2019; 15(1):172-178., Kantner I, Erben RG. Long-term parenteral administration of 2-hydroxypropyl β-cyclodextrin causes bone loss. Toxicol Pathol. 2012; 40(5):742-50.] who experienced progressive cognitive decline in the last year and who meet all of the criteria listed below. Sites aim to enroll roughly equivalent percentages of MCI due to AD and mild AD patients such that there is no more than 60% of either group, although randomization is not constrained on the site level but rather on a study level.

### 7. Eligibility Criteria

Patients are eligible to participate in this study if they meet the inclusion criteria and do not meet any of the exclusion criteria listed below.
I. Inclusion Criteria: Patients are eligible to participate in this study if they meet ALL of the following criteria:
   1. Male or female≥50 to ≤80 years of age, at the time of consent.
      a. Have at least an eighth-grade education.
      b. Primary language for United States-based patients must be English and patients should be able to understand and read English at a level that will ensure adequate completion of study procedures including all cognitive tests.
   2. MCI due to AD (Stage 3) according to the FDA Guidance for Industry on Early Alzheimer's Disease: Developing Drugs for Treatment: [Vellas B, Bateman R, Blennow K, et al. Endpoints for pre-dementia AD trials: a report from the EU/US/CTAD Task Force. J Prev Alzheimers Dis. 2015; 2(2):128-135.]
      a. Meet the NIA-AA criteria for MCI due to AD. [Cummings J. The National Institute on Aging-Alzheimer's Association Framework on Alzheimer's disease: application to clinical trials. Alzheimers Dement. 2019; 15(1):172-178., Kantner I, Erben RG. Long-term parenteral administration of 2-hydroxypropyl β-cyclodextrin causes bone loss. Toxicol Pathol. 2012; 40(5): 742-50.]
      b. Have a global Clinical Dementia Rating (CDR) scale score of 0.5 and a CDR Memory Box score of 0.5 or greater at both Screening (V1) and Baseline (V2).
      c. History of memory complaint/decline reported by patient or partner/caregiver with gradual onset and slow progression over the last 1 year before Screening (V1).
      d. In the opinion of the Investigator based on patient and caregiver review, relatively preserved functional abilities and activities of daily living.
         OR
   Mild AD dementia (Stage 4) according to FDA Guidance for Industry on Early Alzheimer's Disease: Developing Drugs for Treatment: [Vellas B, Bateman R, Blennow K, et al. Endpoints for pre-dementia AD trials: a report from the EU/US/CTAD Task Force. J Prev Alzheimers Dis. 2015; 2(2):128-135.]
      e. Meet the NIA-AA criteria for mild AD dementia. [Cummings J. The National Institute on Aging-Alzheimer's Association Framework on Alzheimer's disease: application to clinical trials. Alzheimers Dement. 2019; 15(1):172-178., Kantner I, Erben R G. Long-term parenteral administration of 2-hydroxypropyl β-cyclodextrin causes bone loss. Toxicol Pathol. 2012; 40(5):742-50.]
      f. Have a global CDR score of 0.5 to 1.0 and a CDR Memory Box score of 0.5 or greater at both Screening (V1) and Baseline (V2).
   3. All patients should have an MMSE-2:SV score≥20 and ≤28 at both Screening (V1) and Baseline (V2) with no more than a 3-point change between visits.
   4. Positive Precivity AD blood test biomarker for AD with high APS (58-100) during the Screening period; or CSF biomarker for supporting the diagnosis of AD.
      a. Only after Precivity AD biomarker or cerebral AD pathology positivity is demonstrated, evidence of cerebral Aβ pathology must also be demonstrated based on CSF analysis evaluating the levels of Aβ42/40 and tau, meeting the criteria of CSF Aβ42/40 ratio<0.72 for patients with MCI and CSF Aβ42/40 ratio<0.72 and a CSF P-tau>50 pg/mL for patients with EAD. The test result to determine cerebral Aβ pathology must be known prior to enrollment at Baseline (V2).
   5. MRI of the brain performed during the screening period or within the prior 12 months that is generally supportive of AD upon local read.
   6. Locally or centrally read MRI of ARIA suggesting less than 4 microhemorrhages.
   7. Have a primary caregiver who has sufficient contact with the patient, is able to provide assessment of cognitive and functional changes and is willing to accept primary responsibility for supervising the patient and assessing the condition of the patient throughout the study in accordance with protocol requirements. The primary caregiver must meet the following criteria:
      a. Be able and willing to provide information needed for efficacy assessments such as CDR-SB.
      b. Willing to sign the caregiver informed consent.
   8. Not likely to experience a change in living conditions (e.g., institutionalization, moving to a different city, etc.) or change in primary caregiver during participation in the trial.
   9. Pro-cognitive drugs or background symptomatic therapy for AD with acetylcholinesterase inhibitors and/or memantine are allowed as long as the dose has been stable for at least 60 days prior to Screening (V1) and is expected to remain stable for the study duration. NOTE: Treatment-naïve patients can be entered into the study but should not begin AD treatment for the duration of the study.
   10. Background medications used for other stable chronic illnesses that are not expressly prohibited are also allowed provided that they remain stable for the study duration:
      a. Psychotropics, such as antidepressants and antipsychotics, must be at a stable dose for at least 30 days prior to Screening (V1) and remain stable throughout the study duration within the framework and regimen in Section 6.7.
      b. Short-acting benzodiazepines for sleep can be used up to 3/week but not within 24 hours of cognitive assessments.
   11. All patients must pass a brief audiologic examination including pure tone audiometry.
   12. Body mass index between 17 and ≤35 kg/m² at Screening (V1), with upper weight limit of 110 kg.
   13. Females participating in the study must meet 1 of the following criteria:
      a. Surgically sterilized (e.g., hysterectomy, bilateral oophorectomy, or tubal ligation) for at least 6 months or postmenopausal (postmenopausal females must have no menstrual bleeding for at least 1 year) or
      b. If not postmenopausal, agree to use highly effective birth control methods including combined (estrogen and progestogen containing) hormonal contraception associated with inhibition of ovulation (oral, intravaginal, or transdermal); progestogen only hormonal contraception associated with inhibition of ovulation (oral, injectable, or implantable); intrauterine device; intrauterine hormone-releasing system; or vasectomized partner, and have negative urine pregnancy test at Screening. Highly effective contraception must be used during the study and until 3 months after the last dose of study treatment.
   14. All sexually active male patients with WOCBP partners (post-menarche) must use a condom with or without spermicide in addition to the birth control used by their partners during the study and until 3 months after the last dose of study treatment.
   15. Age-appropriate estimate glomerular filtration rate calculated by the Modification of Diet in Renal Disease Study.
II. Exclusion Criteria: Patients are not eligible to participate in this study if they meet any of the following exclusion criteria:
   1. Clinically significant renal disease that in the opinion of the Investigator precludes the patient from participating in the study.
   2. Evidence of a neurodegenerative disease other than AD or any medical history or brain imaging abnormality that might cause the dementia in the opinion of the Investigator, including, but not limited to vascular dementia, chronic traumatic encephalopathy, or uncontrolled major depressive disorder (MDD).
   3. Severe hypothyroidism as defined as hypothyroidism that is refractory to standard of care and with no or inadequate response to clinically relevant dosage of levothyroxine as determined by thyroid-stimulating hormone, T3 and T4, and presentation of clinical signs and symptoms.
   4. Abnormally low levels of serum Vitamin B12 (less than lower limit of normal for testing laboratory).
   5. Imaging should be generally supportive of the diagnosis of EAD and, importantly, not consistent with other neurodegenerative disorder or differential dementia diagnoses. For example, any suggestion of vascular disease, including multiple infarctions involving large blood vessels or localized single infarction (angular gyrus, thalamus, anterior cerebral artery and posterior cerebral artery region), multiple lacunae of the basal nuclei or white matter or extensive lesions of the periventricular white matter or combination of several lesions are considered exclusionary. Additionally, any single lacuna in an area known to impact cognition such as the hippocampus will also be exclusionary.
      a. In cases of prior MRI scans used for inclusion, should there be any evidence of new neurological symptoms between that scanning and Screening (V1), rescanning is necessary.
      b. Absence of vascular dementia as defined by a HIS of ≤4 is exclusionary.
   6. Lacks visual, auditory acuity and/or language abilities adequate to perform cognitive assessments.
   7. History of any medical illness, such as cancer, requiring systemic therapy in the last 5 years, except for localized basal cell carcinoma of the skin and in-situ cervical cancer successfully treated with surgical excision; history of severe heart failure (Grade 2 or higher on the New York Heart Association scale), major stroke, uncontrolled seizure disorder, or other medical illnesses that in the Investigator's opinion will increase the patient's risk of participation in the study or confound study assessments.
   8. Patients with the following measurements identified during Screening:
      a. Platelet count<50,000/μL.
      b. International normalized ratio>1.5.
      c. Patients with a hemorrhagic trend (activated partial thromboplastin time>1.5 upper limit of normal).
9. Positive blood screen for human immunodeficiency virus (HIV-1 and HIV-2), hepatitis B surface antigen, or hepatitis C virus antibodies at Screening (V1) unless successful curative treatment for hepatitis C (e.g., Harvoni) has been received and there is documentation that there is no hepatitis B/C virus detected 3 months after completion of treatment.
10. Any planned surgery that requires a general anesthesia that would take place during the study. Local anesthesia during outpatient surgery is permitted if, in the opinion of the Investigator, this operation does not interfere with study procedures and patient safety.
11. History or current evidence of major psychiatric illness such as schizophrenia, bipolar disorder, or MDD per Diagnostic and Statistical Manual of Mental Disorders 5th Edition (DSM-5); or MDD will be excluded if GDS-SF score>8 at Screening (V1).
12. History of violent or aggressive behavior that requires medication to control.
13. Suicidal thoughts, intentions, or actions captured during screening by C-SSRS or history of suicidal attempt in 2 years prior to Screening (V1). NOTE: Should not have had active suicidal thoughts (Type 4 or 5 on the C-SSRS) in the 6 months preceding Screening (V1) or at Baseline (V2); or have a history of a suicide attempt in the previous 2 years; or be at serious suicide risk in the Investigator's clinical judgment.
14. History of alcohol or drug abuse or dependence within 12 months of screening as defined by the DSM-5. NOTE: Patients will be excluded if they have a positive urine screen for drugs of abuse that include methadone, cocaine, and amphetamines; positive urine screen for opiates, barbiturates, or benzodiazepines without a prescription at Screening (V1).
15. Previous or ongoing treatment with Aduhelm, donanemab, or investigational AD vaccine therapy for amyloid or tau; or participation in a clinical study involving monoclonal antibodies, or derivatives, or immunoglobulins within 12 months before Screening (V1) unless it can be documented that the patient was randomized to placebo.
16. Has been treated with any known drugs that are moderate or strong inhibitors/inducers of cytochrome P450 enzymes (e.g., barbiturates, phenothiazines, cimetidine, carbamazepine) within 30 days before the first dose of study medication, and that, in the Investigator's judgment, may impact patient safety or the validity of the study results.
17. Has any of the following otologic-related exclusion criteria:
   a. Current use, or within 60 days prior to study enrollment, of ototoxic medications, including, but not limited to, aminoglycoside antibiotics (gentamicin, tobramycin, amikacin, streptomycin); platinum-containing chemotherapies (cisplatin, carboplatin, oxaliplatin); or loop diuretic (furosemide).
   b. History of idiopathic sensorineural hearing loss, otosclerosis, or vestibular schwannoma.
   c. History of middle ear or inner ear surgery or middle ear effusion.
      i. Medical history of recurrent otitis media that in the opinion of the Investigator could impact patient eligibility.
   d. Current conductive or sensorineural hearing loss (a Grade 3 hearing loss is observed, as defined by >25 decibel loss at 3 successive frequencies between 1 kHz and 8 kHz) unilaterally or bilaterally.
18. QTc prolongation risk such as a history of additional risk factors for TdP or sudden death (e.g., heart failure, hypokalemia, family history of Long QT Syndrome, unexplained syncope, concurrent medications including anti-depressant agents known to prolong QT/QTc.

### 8. Study Drugs

Trappsol® Cyclo™ (obtained from Cyclo Therapeutics, Inc.) and placebo (0.45% sodium chloride for intravenous infusion as control [½ normal saline]).

### 9. Safety Endpoints

Incidence and severity of TEAEs.

Adverse events of special interest (AESIs): (1) MRI assessments of amyloid-related imaging abnormalities-hemorrhagic (ARIA-H) or amyloid-related imaging abnormalities-edematous (ARIA-E), (2) hearing loss based audiological examination, and (3) infusion reactions.

Vital signs and/or physical examinations.

Safety clinical laboratory values (hematology, clinical chemistry, and urinalysis) including C-terminal telopeptide.

Triplicate 12-lead electrocardiogram (ECG) to support cardiodynamic evaluation, including concentration-QTc analysis. Triplicate ECGs will be time-matched with PK samples at Week 0 (Baseline), Week 4, and Week 8.

Columbia-Suicide Severity Rating Scale (C-SSRS).

### 10. Secondary Endpoints

Mean change in total ADAS-Cog-14 score from Baseline (V2) to Weeks 12 (V5) and 24 (V8).

Change in CDR-SB from Baseline (V2) to Weeks 12 (V5) and 24 (V8).

Change in MMSE-2:SV total score from Baseline (V2) to Weeks 12 (V5) and 24 (V8).

Change in ADCS-CGIC from Baseline (V2) to Weeks 12 (V5) and 24 (V8).

Change in ADCS-ADL from Baseline (V2) to Weeks 12 (V5) and 24 (V8).

### 11. Exploratory Endpoints

Change in combined Z-scores from Baseline (V2) to Weeks 12 (V5) and 24 (V8) on various outcome measures and cognitive domains: (1) ADAS-Cog-14, (2) CDR-SB, and (3) MMSE-2:SV.

Pharmacodynamic blood/plasma evaluation of AB, Aβ42, tau, total tau, phosphorylated tau, 24S-hydroxycholesterol, neuronal extracellular vesicle biomarker, neurogranin, NfL, and other disease-related biomarkers.

### 12. Pharmacokinetic Endpoints

PK parameters including but not limited to Cmax, Tmax, half-life, AUClast, AUCinf, T½, Clast, and Tlast (see Table 10.1 in main protocol) are calculated. Plasma levels are measured prior to the start of the study drug infusion, 1 and 3 hours into the infusion, upon completion of infusion, 50 to 60 minutes post-infusion, and 2 hours post-infusion at Baseline (Week 0 (V2), Week 4 (V3), Week 8 (V4). Plasma samples are collected for potential analysis of PK trough levels will be measured prior to start of study drug infusion at Week 12 (V5) and Week 24 (V8).

### 13. Statistical Procedures

Descriptive statistics for ordinal (e.g., Likert scale) and continuous variables include the number of patients with non-missing values, mean, median, standard deviation, minimum value, and maximum value.

Safety analyses are based on the Safety Population, defined as all randomly assigned patients who receive at least 1 dose of study drug. Efficacy analyses use the Full Analysis Set, which is based on the Intent-to-Treat principle.

For each efficacy endpoint, change from baseline to each applicable post-Baseline visit is assessed for treatment group differences using a mixed model for repeated measures with factors for study site, treatment, week, and the treatment-by-week interaction, and using the baseline response variable value as a covariate.

As a sensitivity analysis, an analysis of covariance with fixed factors for study site and treatment, with baseline value as a covariate, is used.

Additionally, subgroup analyses may be performed. The subgroup analyses may include but are not limited to the presence or absence of ongoing approved AD treatment (e.g., acetylcholinesterase inhibitors or memantine or both) and ApoE4 status (i.e., ApoE4 positive or negative) or other groupings of patients.

AEs are categorized by system organ class and preferred term with the Medical Dictionary for Regulatory Activities. Summary tables for TEAEs include number and percent of patients experiencing TEAEs by system organ class and preferred term.

Mean change in safety clinical laboratory parameters (hematology, clinical chemistry, and urinalysis), including C-terminal telopeptide, and vital signs from baseline to each post-Baseline visit are summarized descriptively. Safety clinical laboratory results considered clinically important by the Investigator are identified.

The planned statistical analysis methods are described in more detail in the Statistical Analysis Plan, which are finalized prior to database lock.

### INCORPORATION BY REFERENCE

The entire disclosure of each of the patent documents and scientific articles referred to herein is incorporated by reference for all purposes.

### EQUIVALENTS

The disclosure can be embodied in other specific forms with departing from the essential characteristics thereof. The foregoing embodiments therefore are to be considered illustrative rather than limiting on the disclosure described herein. The scope of the disclosure is indicated by the appended claims rather than by the foregoing description, and all changes that come within the meaning and range of equivalency of the claims are intended to be embraced therein.

1. A method of treating early Alzheimer's disease in a human patient in need thereof, the method comprising administering to the human patient an effective amount of a hydroxypropyl β-cyclodextrin composition.

2. The method of claim 1, wherein the early Alzheimer's disease is Alzheimer's disease with mild cognitive impairment or mild Alzheimer's disease.

3. The method of claim 1, wherein at the time of initiation of administration of the effective amount of the hydroxypropyl β-cyclodextrin composition the human patient exhibits progressive cognitive decline.

4. The method of claim 1, wherein at the time of initiation of administration of the effective amount of the hydroxypropyl β-cyclodextrin composition the human patient has exhibited progressive cognitive decline for at least about 1 year.

5-8. (canceled)

9. The method of claim 1, wherein the human patient has previously been administered a pro-cognitive drug and/or a symptomatic therapy for early Alzheimer's disease.

10. The method of claim 1, wherein the human patient has previously been administered an acetylcholinesterase inhibitor and/or memantine.

11. The method of claim 1, wherein the human patient is at least 50 years old.

12.-14. (canceled)

15. The method of claim 1, wherein administering an effective amount of the hydroxypropyl β-cyclodextrin composition comprises administering to the human patient about 500 mg/kg to about 2000 mg/kg of the hydroxypropyl β-cyclodextrin composition.

16. (canceled)

17. The method of claim 1, wherein administering an effective amount of the hydroxypropyl β-cyclodextrin composition comprises administering to the human patient by intravenous infusion about 500 mg/kg to about 2000 mg/kg of the hydroxypropyl β-cyclodextrin composition.

18. (canceled)

19. The method of claim 1, wherein administering an effective amount of the hydroxypropyl β-cyclodextrin composition comprises administering to the human patient by intravenous infusion about 500 mg/kg to about 2000 mg/kg of the hydroxypropyl β-cyclodextrin composition, every 28 days.

20. (canceled)

21. The method of claim 1, wherein administering an effective amount of the hydroxypropyl β-cyclodextrin composition comprises administering to the human patient by intravenous infusion about 500 mg/kg to about 2000 mg/kg of the hydroxypropyl β-cyclodextrin composition, every 28 days, for a period of at least about 24 weeks.

22. (canceled)

23. The method of claim 1, wherein the hydroxypropyl β-cyclodextrin composition is administered to the human patient by intravenous infusion over a period of at least about 4 hours.

24. (canceled)

25. The method of claim 1, wherein the hydroxypropyl β-cyclodextrin composition comprises about 25% (w/v) of a hydroxypropyl β-cyclodextrin.

26. The method of claim 1, wherein the hydroxypropyl β-cyclodextrin composition comprises a mixture of two or more hydroxypropyl β-cyclodextrin species.

27. (canceled)

28. The method of claim 26, wherein the mixture of the two or more hydroxypropyl β-cyclodextrin species has a molar substitution value from about 0.59 to about 1.14.

29-30. (canceled)

31. The method of claim 1, wherein the hydroxypropyl β-cyclodextrin composition comprises about 0.2% w/w or less of unsubstituted β-cyclodextrin.

32. (canceled)

33. The method of claim 1, wherein the hydroxypropyl β-cyclodextrin composition comprises about 2.5% (w/w) or less of propylene glycol.

34. The method of claim 1, further comprising administering a second therapeutic agent selected from the group consisting of donepezil, rivastigmine, galantamine, memantine, verubecestat, solanezumab, bapineuzumab, aducanumab, tideglusib, epothilone D and ABBV-8E12.

35. The method of claim 1, further comprising administering a second therapeutic agent selected from the group consisting of a cholinesterase inhibitor, an NMDA receptor antagonist, a humanized antibody which targets tau protein, a humanized antibody which targets amyloid beta protein, and a BACE inhibitor.

36. (canceled)
